(19) United States
(12) Patent Application Publication
(10) Pub. No.: US 2008/0103460 A1
(43) Pub. Date: May 1, 2008

(54) Method for making an appliance for delivering a composition, the appliance having an elastic layer and a shielding layer

(76) Inventors: Kenneth B. Close, New London, WI (US); Jonathan K. Arendt, Appleton, WI (US); Gary V. Anderson, Larsen, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/590,443

(22) Filed: Oct. 31, 2006

(51) Int. Cl. A61M 35/00

(52) U.S. Cl. 604/292; 604/293

(57) ABSTRACT

Disclosed is a method for making an appliance adapted to transfer to, or associate with, the skin or tissue of a user, a composition, e.g., a moisturizing formulation. Because of possible interactions between the formulation and/or formulation ingredients and elastic polymers that may be employed to help give the appliance elastic qualities, the method comprises steps for providing an elastic layer or member and a shielding layer or member, with the shielding member interposed between the composition/formulation and the elastic member. The shielding layer and elastic layer may be integrally connected to one another, as in a film comprising two layers. Typically the shielding layer will be impermeable to the composition/formulation (e.g., by employing polymers, such as polypropylene comprising crystalline portions, such that the mass transport of the formulation through the shielding member is stopped or substantially impeded).

### BACKGROUND

People rely on various kinds of formulations or compositions for health and/or hygiene benefits.

Generally, two categories of formulations are used when moisturizing and/or hydrating skin. Humectants are used to transport moisture from the environment (primarily water vapor in air) to skin. One example of a humectant is glycerin. Such materials tend to be hydrophilic (i.e., water loving), and are generally non-greasy, light in weight and/or viscosity, and quick to apply. Occlusive materials are used to trap moisture already present in the skin. These materials tend to create a water barrier over the skin, thereby inhibiting the transport of water from the skin to the external environment. An example of an occlusive material is petrolatum. Such materials tend to be heavier, effective over longer periods of time, and often contain oily and/or greasy ingredients.

Often people apply such formulations directly to skin using their hands. If the formulation includes water as an ingredient, water may evaporate, potentially decreasing the effectiveness of the formulation. Furthermore, after application of the formulation to the body, any excess formulation remaining on the hands must be removed.

In some instances, gloves, socks, sleeves, or other appliances have been used in conjunction with formulations. For example, a user either applies a formulation to his or her hand or foot, and then dons or applies a glove over the treated hand or a sock over the treated foot. Alternatively, a user slips on a glove or sock that has been pre-treated with a skin-care formulation. Unfortunately, such items have typically been made of a polymeric material (e.g., neoprene rubber) lacking a cloth-like appearance and/or feel. Often such items do not conform readily to the complex surfaces and contours of a foot or hand.

One alternative is described in co-pending U.S. patent application Ser. No. 11/190,597, entitled “Appliance for Delivering a Composition” to K. Close, et al., which is hereby incorporated by reference in its entirety in a manner consistent herewith. A liquid-impermeable layer, such as a film, is sandwiched between two fibrous layers, such as nonwovens, when making, for example, a moisturizing sock, glove, sleeve, or other appliance. A composition is associated with the interior of the appliance, and is adapted to be transferred from the appliance to the skin of a user of the appliance (e.g., to moisturize the skin of a hand of a user of a moisturizing glove).

If a film comprises elastomeric ingredients (e.g., KRATON polymers; such as Kraton 6638 polymer resin [Kraton 6638 is a blend of 80% by weight Kraton 1730 styrene-(ethylene-propylene)-styrene-(ethylene-propylene) tetrablock copolymer from Kraton Polymers LLC, 7% by weight PETROTHANE NA601 polyethylene wax from Quantum Chemical Co., and 13% by weight REGALREZ 1126 tackifier from Eastman Chemical Co.]; a tackifier and/or wax need not be employed), these ingredients help produce elastomeric qualities in the resulting film. Accordingly, the appliance itself can have elastomeric qualities, which can assist in producing an appliance with improved fit and/or contact between the inner surface of the appliance and the skin of the wearer of the appliance. Some ingredients in a skin-care composition, however, may interact with or degrade ingredients in, or be adsorbed or absorbed into, the elastomeric layer (e.g., oily substances from a formulation can react with, partially solubilize, adsorb or absorb onto, or otherwise interact with elastomeric materials, such as the KRATON polymers referred to above). Such interactions may result in changes to chemical and/or physical properties in the elastomeric layer. Furthermore, these skin-care ingredients may also degrade or interact with components of various adhesive compositions, if such compositions are used to help make the appliance. In effect, the choice of formulation/composition ingredients, or the design parameters of the appliance, may be constrained by such interactions between appliance materials and formulation ingredients.

What is needed is an appliance, and method for making said appliance, that promotes health and/or hygiene by facilitating transport of a formulation or composition to, and/or contact with, skin or tissue, and which: conforms readily to the contours and surfaces of parts of the body to which the appliance is applied; and which reduces or minimizes undesirable interactions between a formulation and/or formulation ingredients, and an elastic member or layer (such as the aforementioned elastomeric film/liquid-impermeable layer).

### SUMMARY

We have determined that an appliance, such as a sleeve, sock, or glove, that comprises an elastic member or layer, and a shielding member or layer that helps shield the elastic member from undesired interactions with a formulation and/or one or more ingredients in the formulation, effectively and comfortably treats the skin or tissue of a user. Humectants, materials of an occlusive nature, and numerous other ingredients may be included in the formulation, examples of which are provided below in the Description section.

In one embodiment of the present invention, there is disclosed a method for making an appliance that comprises a multi-layer film in which a first layer, which is adapted to be adjacent to a formulation, has one or more polymers with a higher degree of crystallinity than one or more polymeric ingredients in a second layer that imparts elastomeric qualities to that second layer, i.e., the first layer of the film acts as a shielding member, and the second layer acts as an elastic member. For example, one version of a method of the present invention results in an appliance comprising a two-layer film, a first layer adjacent to (which, for purposes of this application, means next, proximate, or close to) the formulation comprises one or more polymers having a higher degree of crystallinity than the elastomeric material(s) in a second layer that is attached to the first layer. In this way the first layer or shielding member helps reduce transport or diffusion of ingredients from the formulation through the shielding member and to the second layer or elastic member, where such ingredients can potentially interact in a negative way with one or more elastomeric polymers in said second layer. Without being bound to a particular theory, a first layer or shielding member having a more crystalline nature than the second layer or elastic member can resist or reduce passage of molecules comprising carbon chains (e.g., oils, fatty-acid molecules, etc.) that may be capable of interacting with amorphous portions of an elastomeric polymer, which typically are carbon chains themselves (and/or which may be adsorbed or absorbed into the elastomeric layer or member). A formulation may then be associated with the shielding member by, e.g., coating, spraying, printing, brushing, depositing, injecting, etc. the formulation on or in the shielding member. The formulation need not be uniformly deposited or associated with the entire shielding member. If desired, the formulation can be associated with discrete locations on the shielding member. Or a plurality of different formulations may be associated with different locations on the shielding member/layer. Note too that often the shielding layer or member is substantially impermeable to the formulation itself.

In one embodiment of the present invention, a multi-layer film (such as the two-layer film described above), is made by co-extruding said layers when making the film.

In another embodiment of the present invention, the multi-layer film is attached to an outer fibrous substrate. This outer substrate can impart a cloth-like appearance and feel to the appliance. In one version of the present invention, the outer fibrous substrate is attached to the film at discrete points or regions (as with, e.g., thermal point bonding). In another version of the present invention, the film is in a stretched condition when the outer fibrous substrate is bonded to it (e.g., at discrete points or regions), and then allowed to retract, thereby helping effect an increased rugosity or increase in undulations associated with the outer fibrous layer.

In another embodiment of the present invention, the multi-layer film is attached to an inner fibrous substrate. This inner substrate can help contain the formulation or composition associated with the appliance, and can help reduce leaking of said formulation or composition. In one version of the present invention, the inner fibrous substrate is attached to the film at discrete points or regions (as with, e.g., thermal point bonding). In another version of the present invention, the film is in a stretched condition when the inner fibrous substrate is bonded to it (e.g., at discrete points or regions), and then allowed to retract, thereby helping effect an increased rugosity or increase in undulations associated with the inner fibrous layer. For those versions of the appliance which comprise an inner fibrous layer, the composition may be associated with the inner fibrous layer of the appliance.

In another embodiment of the present invention, the multi-layer film is attached to both an inner fibrous substrate and an outer fibrous substrate as described in the preceding paragraphs. It should be noted that, for those versions of the present invention where the shielding member and elastic member are co-extruded layers and intimately attached to one another, the elastomeric qualities of the elastic member will be somewhat limited by the nature of the shielding member (i.e., the shielding member, which comprises crystalline polymers or polymer segments, will likely be less elastic than the elastic member).

In another version of the present invention, there is disclosed a method for making an appliance that comprises a film comprising an elastomeric polymer (i.e., an elastic member), and a separate layer comprising a polymer having a higher degree of crystallinity than said elastomeric polymer. The layers are bonded or fused together to form the appliance. The appliance employs the layer comprising a polymer having a higher degree of crystallinity as a shielding member so that any formulation ingredient that might undesirably interact with one or more elastomers must first pass through the layer with the more crystalline polymer. Without being bound to a particular theory, the increased crystallinity of the shielding member reduces the rate of mass transport or diffusion of formulation ingredients through said shielding layer, thereby reducing or minimizing the amount of formulation ingredient reaching, and interacting with, elastomeric polymers in the film (and/or being absorbed or adsorbed by the elastomeric film). As noted above, the formulation or composition is associated with the film layer acting as a shielding member. Furthermore, in some versions of the invention, the shielding member is substantially impermeable to the formulation itself.

In other versions of the present invention, the aforementioned elastomeric film and so-called “shielding” layer comprising a polymer of higher crystallinity than one or more elastomeric polymers in the elastomeric film are attached to an outer fibrous layer, an inner fibrous layer, or both (as described in preceding paragraphs). As noted in preceding paragraphs, if an inner fibrous layer is being employed, then the formulation or composition will typically be associated with the inner fibrous layer.

For those embodiments where one layer, such as a single-layer film, is to be stretched during manufacture of the appliance, it should be noted that there is an advantage to decoupling, at least in part, the elastomeric function of the appliance from the barrier function of the appliance. For example, if a single layer, such as a film, was to provide the appliance with the ability to stretch in both the machine direction (MD) and the cross-machine direction (CD), this may be thought of as providing an elastomeric function to the appliance. (Note: The direction of travel of a web, such as a nonwoven web, along a production machine is known as the machine direction; and the direction transverse to the machine direction—i.e., across the width of the web—is known as the cross-machine direction.) And if this same layer was to be impermeable to liquid or water, so that a formulation associated with the interior of the appliance does not leak through the appliance during use, then the same layer is also providing a barrier function for the appliance. Thus the polymeric constituents of this single layer must be chosen to provide, if desired in the appliance, both elastomeric qualities and barrier qualities. This may prove difficult. Generally polymeric constituents that provide an elastomeric quality are somewhat amorphous, and these amorphous regions typically comprise carbon chains. If this same film is also the only component of the appliance adapted to be a liquid- or water-impermeable material (i.e., act as a barrier to the formulation—meaning that the formulation, during shipping, storage, and subsequent use, does not diffuse or transfer to the outside of the appliance in significant quantities such that a user of the appliance, when contacting other surfaces, transfers noticeable quantities of the formulation from the exterior of the appliance to the other surfaces), then the film will also likely contact formulation associated with the interior of the appliance. Accordingly, certain formulation ingredients, such as oils, may tend to break down or alter those polymeric constituents that are more amorphous (such as polymeric constituents used to impart elastomeric qualities to an article in which the polymeric constituents are employed). These ingredients may also adsorb or absorb on or in the elastomeric layer, degrading the physical and/or chemical properties of said layer.

By providing an appliance that comprises both an elastic member and a shielding member, the polymeric constituents and other materials may be selected for each member so that it better performs its primary function, i.e., the materials need not be chosen so that, in one uniform layer, the elastomeric function and barrier function are satisfactorily performed. As noted above, the polymeric constituents for the shielding member may be chosen such that they have a higher degree of crystallinity, because a primary function of the shielding member is to act as a barrier to the formulation and/or certain ingredients in the formulation (e.g., oil-like substances). And the materials used to make the elastic member may be chosen to better effect the elastomeric properties of the elastic member. By interposing the shielding member between the elastic member and a formulation in accordance with the present invention, polymeric constituents for the elastic member, which may be amorphous, can be used with a lessened risk of degradation from ingredients in the formulation (because the ingredients are less likely to diffuse or otherwise transfer across the shielding member—e.g., due to polymers of higher crystallinity being used in the shielding member).

The decoupling of the barrier function from the shielding function can also facilitate manufacture of an appliance of the present invention (including, for example, the ability to select porous webs to provide an elastomeric quality to the web—because another layer acts as a shielding member/provides a barrier function to the appliance). If, for example, an inner and/or outer layer (e.g., a nonwoven facing) is to be attached to a single-layer, unstratified film when the film is in a stretched condition, then the film will provide both an elastomeric function and a barrier function. As noted above, its chemical makeup must be balanced to effect both of these qualities.

In some methods of the present invention, individual elastic components (e.g., elastic strands) comprising one or more elastomeric polymers impart an elastomeric quality to the appliance. Because the elastomeric function of the appliance is decoupled from the barrier function of the appliance, individual elastic components (e.g., a web in which individual elastic strands are extruded in a spaced-apart, substantially parallel fashion—with these strands attached to a web of meltblown fiber, thereby forming an elastomeric composite that may be liquid-permeable) may be employed in the elastic member. Because a shielding member is interposed between an elastic member/layer and the skin-care formulation or composition that is associated with the appliance: (1) the elastic member/layer may be porous/liquid-permeable; and (2) the polymeric constituents of the elastic member may be selected to help effect the elastomeric function of this member, without having to balance the elastomeric function with the barrier function of the appliance.

Another advantage of decoupling the barrier function and elastomeric function of the appliance is that the elastomeric member can be stretched during manufacture of the appliance (e.g., so that other layers may be bonded to the stretched elastomeric member at discrete locations; with the elastomeric member then being allowed to relax, thereby gathering the other layers and producing an undulating surface), while the shielding member is not stretched, or is stretched less, during manufacture.

So, for example, in one version of the invention, the elastomeric member is stretched prior to its being directed to a nip between two rolls. The elastomeric member/layer could be a film, individual elastic components (e.g., a composite comprising spaced-apart elastic strands attached to meltblown fiber), or some other elastomeric substrate. The shielding member/layer can be, for example, a film comprising one or more crystalline polymers, or polymers having crystalline portions, such that oil-like or other ingredients in a formulation do not readily diffuse through the shielding layer within the time between manufacture and use of the appliance, with the film directed to the same nip, but in an unstretched condition. By discretely bonding the unstretched shielding member (e.g., a film) to the stretched elastomeric member at the nip (e.g., by bonding the shielding member and elastomeric member at discrete locations along their lengths), and subsequently allowing the elastomeric member to retract to its unstretched state after this attachment, then an appliance substrate can be produced in which the shielding member has an undulating, hilly shape (i.e., at certain points along its length the shielding member is attached, and next to, the elastomeric member; between these points of attachment the shielding member rises and falls—imagine a hill—because between the points of attachment the shielding member is unattached to the elastomeric member). In the finished appliance, the elastomeric member can be stretched to help fit the appliance to the part of the body with which the appliance is being used (e.g., a sock or glove for transferring a composition to a hand or foot). While the shielding member lacks the same ability to stretch and retract that the elastomeric member possesses, the fact that the shielding member is gathered (i.e., there is excess, unattached material between the points of attachment) means that when the elastomeric web is stretched, the shielding member does not restrain the stretching of the appliance substrate as a whole.

Note that the preceding paragraph refers to stretching of the appliance substrate in a direction that is similar to the machine-direction of the substrate during its manufacture. What about the cross-machine direction? The elastomeric member, by its nature, provides some ability to stretch in the cross-machine direction. The shielding member can be made or made such that it provides some ability to stretch, and retract to its original shape without a significant loss its original dimensions, in the cross-machine direction. For example, a shielding member can be a laminate of a film comprising crystalline polymer(s) and/or crystalline polymer segments such that mass transport of the formulation and its components is reduced or stopped (at least for the duration typifying the time from manufacture to use, which may be, for example, from about 1 to about 8 months)—the film possessing some ability to stretch in the cross-machine direction; and a necked spunbonded facing oriented so that it to, after being attached to the film, is also capable of stretching in the cross-machine direction.

Typically the resulting appliance will be adapted for limited use, suitably a single use.

Furthermore, the resulting appliance will usually have a formulation associated with the appliance. Nevertheless, in some versions of the invention, the appliance is not pre-coated/treated with a formulation, thereby allowing a user of the appliance the option of choosing a formulation to apply to his or her skin, and then donning (as with, e.g., a glove, sock, or sleeve) or affixing (as with a patch) an appliance over at least a portion of the skin to which the formulation was applied.

These and other versions of the invention are described more fully below.

### DRAWINGS

FIG. 1A representatively illustrates one version of a substrate of that may be made by a method of the present invention.

FIG. 1B representatively illustrates one version of a substrate that may be made by a method of the present invention.

FIG. 1C representatively illustrates one version of a substrate that may be made by a method of the present invention.

FIG. 1D representatively illustrates one version of a substrate that may be made by a method of the present invention.

FIG. 2 representatively illustrates one version of a substrate that may be made by a method of the present invention and cut so that the substrate's perimeter defines the shape of a hand.

FIG. 2A representatively illustrates one version of an appliance that may be made by a method of the present invention.

FIG. 3 representatively illustrates one version of a substrate that may be made by a method of the present invention and cut so as to form a foot appliance of the present invention.

FIG. 3A representatively illustrates one version of an appliance that may be made by a method of the present invention.

FIG. 4 representatively illustrates one version of a method of the present invention.

FIG. 5 representatively illustrates one version of a method of the present invention.

### DEFINITIONS

Within the context of this specification, each term or phrase below includes the following meaning or meanings:

“Attach” and its derivatives refer to the joining, adhering, connecting, bonding, sewing together, depositing on, associating with, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Coform” refers to a blend of meltblown fibers and absorbent fibers such as cellulosic fibers that can be formed by air forming a meltblown polymer material while simultaneously blowing air-suspended fibers into the stream of meltblown fibers. The coform material may also include other materials, such as superabsorbent materials. The meltblown fibers and absorbent fibers are collected on a forming surface, such as provided by a foraminous belt. The forming surface may include a gas-pervious material that has been placed onto the forming surface.

“Composition,” “formulation,” or their derivatives, when used in the context of a material applied or deposited on the interior surface of an appliance of the present invention (or when applied separately to skin or tissue, with a user then donning or affixing the appliance to the skin or tissue to which the formulation/composition was applied), refers to the various materials that help improve the health and/or hygiene of a user of the appliance.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

“Disposable” refers to articles which are designed to be discarded after a limited use rather than being laundered or otherwise restored for reuse.

The terms “disposed on,” “disposed along,” “disposed with,” or “disposed toward” and variations thereof are intended to mean that one element can be integral with another element, or that one element can be a separate structure bonded to or placed with or placed near another element.

“Fiber” refers to a continuous or discontinuous member having a high ratio of length to diameter or width. Thus, a fiber may be a filament, a thread, a strand, a yarn, or any other member or combination of these members.

“Formulation impermeable,” or variations thereof, when used in describing a layer or multi-layer laminate means that a formulation, such as a formulation adapted to moisturize skin, will not pass to any appreciable extent through the layer or laminate, under ordinary use conditions, in a direction generally perpendicular to the plane of the layer or laminate at the point of formulation contact.

“Formulation permeable,” or variations thereof, refers to any material that is not formulation impermeable.

“Hydrophilic” describes materials or surfaces which are wetted by aqueous liquids in contact with the material or surface. The degree of wetting of the material or surface can, in turn, be described in terms of the contact angles and the surface tensions of the liquids and materials (or surfaces) involved.

“Layer” when used in the singular can have the dual meaning of a single element or a plurality of elements.

“Liquid impermeable,” when used in describing a layer or multi-layer laminate means that liquid, such as water, will not pass to any appreciable extent through the layer or laminate, under ordinary use conditions, in a direction generally perpendicular to the plane of the layer or laminate at the point of liquid contact.

“Liquid permeable” refers to any material that is not liquid impermeable.

“Meltblown” refers to fibers formed by extruding a molten thermoplastic material through a plurality of fine, usually circular, die capillaries as molten threads or filaments into converging high velocity gas (e.g., air) streams, generally heated, which attenuate the filaments of molten thermoplastic material to reduce their diameters. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblowing processes can be used to make fibers of various dimensions, including macrofibers (with average diameters from about 40 to about 100 microns), textile-type fibers (with average diameters between about 10 and 40 microns), and microfibers (with average diameters less than about 10 microns). Meltblowing processes are particularly suited to making microfibers, including ultra-fine microfibers (with an average diameter of about 3 microns or less). A description of an exemplary process of making ultra-fine microfibers may be found in, for example, U.S. Pat. No. 5,213,881 to Timmons, et al. Meltblown fibers may be continuous or discontinuous and are generally self bonding when deposited onto a collecting surface.

“Member” when used in the singular can have the dual meaning of a single element or a plurality of elements.

“Nonwoven” and “nonwoven web” refer to materials and webs of material that are formed without the aid of a textile weaving or knitting process. For example, nonwoven materials, fabrics or webs have been formed from many processes such as, for example, meltblowing processes, spunbonding processes, air laying processes, and bonded carded web processes.

“Spunbonded fibers” refers to small diameter fibers which are formed by extruding molten thermoplastic material as filaments from a plurality of fine, usually circular capillaries of a spinneret with the diameter of the extruded filaments then being rapidly reduced to fibers as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al., the contents of which are incorporated herein by reference in their entirety. Spunbond fibers are generally continuous and have diameters generally greater than about 7 microns, more particularly, between about 10 and about 20 microns.

“Stretch bonded laminate” refers to a composite material having at least two layers in which one layer is a gatherable layer and the other layer is an elastic layer. The layers are joined together when the elastic layer is extended from its original condition so that upon relaxing the layers, the gatherable layer is gathered. Such a multilayer composite elastic material may be stretched to the extent that the nonelastic material gathered between the bond locations allows the elastic material to elongate. One type of stretch bonded laminate is disclosed, for example, by U.S. Pat. No. 4,720,415 to Vander Wielen et al., the content of which is incorporated herein by reference in its entirety. Other composite elastic materials are disclosed in U.S. Pat. No. 4,789,699 to Kieffer et al., U.S. Pat. No. 4,781,966 to Taylor and U.S. Pat. Nos. 4,657,802 and 4,652,487 to Morman and U.S. Pat. No. 4,655,760 to Morman et al., the contents of which are incorporated herein by reference in their entirety.

“Necking” or “neck stretching” interchangeably refer to a method of elongating a nonwoven fabric, generally in the machine direction, to reduce its width (cross-machine direction) in a controlled manner to a desired amount. The controlled stretching may take place under cool, room temperature or greater temperatures and is limited to an increase in overall dimension in the direction being stretched up to the elongation required to break the fabric, which in most cases is about 1.2 to 1.6 times. When relaxed, the web retracts toward, but does not return to, its original dimensions. Such a process is disclosed, for example, in U.S. Pat. No. 4,443,513 to Meitner and Notheis, U.S. Pat. Nos. 4,965,122, 4,981,747 and 5,114,781 to Morman and U.S. Pat. No. 5,244,482 to Hassenboehier Jr. et al., the contents of which are incorporated herein by reference in their entirety.

“Necked material” refers to any material which has undergone a necking or neck stretching process.

“Reversibly necked material” refers to a material that possesses stretch and recovery characteristics formed by necking a material, then heating the necked material, and cooling the material. Such a process is disclosed in U.S. Pat. No. 4,965,122 to Morman, commonly assigned to the assignee of the present invention, and incorporated by reference herein in its entirety. As used herein, the term “neck bonded laminate” refers to a composite material having at least two layers in which one layer is a necked, non-elastic layer and the other layer is an elastic layer. The layers are joined together when the non-elastic layer is in an extended (necked) condition. Examples of neck-bonded laminates are such as those described in U.S. Pat. Nos. 5,226,992, 4,981,747, 4,965,122 and 5,336,545 to Morman, the contents of which are incorporated herein by reference in their entirety.

“Stitchbonded” refers to a process in which materials (fibers, webs, films, etc.) are joined by stitches sewn or knitted through the materials. Examples of such processes are illustrated in U.S. Pat. No. 4,891,957 to Strack et al. and U.S. Pat. No. 4,631,933 to Carey, Jr., the contents of which are incorporated herein by reference in their entirety.

“Ultrasonic bonding” refers to a process in which materials (fibers, webs, films, etc.) are joined by passing the materials between a sonic horn and anvil roll. An example of such a process is illustrated in U.S. Pat. No. 4,374,888 to Bornslaeger, the content of which is incorporated herein by reference in its entirety.

“Thermal point bonding” involves passing materials (fibers, webs, films, etc.) to be bonded between a heated calender roll and an anvil roll. The calender roll is usually, though not always, patterned in some way so that the entire fabric is not bonded across its entire surface, and the anvil roll is usually flat. As a result, various patterns for calender rolls have been developed for functional as well as aesthetic reasons. Typically, the percent bonding area varies from around 10 percent to around 30 percent of the area of the fabric laminate. As is well known in the art, thermal point bonding holds the laminate layers together and imparts integrity to each individual layer by bonding filaments and/or fibers within each layer.

“Elastic” refers to any material, including a film, fiber, nonwoven web, or combination thereof, which upon application of a biasing force in at least one direction, is stretchable to a stretched, biased length which is at least about 110 percent, suitably at least about 130 percent, and particularly at least about 150 percent, its relaxed, unstretched length, and which will recover at least 15 percent of its elongation upon release of the stretching, biasing force. In the present application, a material need only possess these properties in at least one direction to be defined as elastic.

“Extensible and retractable” refers to the ability of a material to extend upon stretch and retract upon release. Extensible and retractable materials are those which, upon application of a biasing force, are stretchable to a stretched, biased length and which will recover a portion, preferably at least about 15 percent, of their elongation upon release of the stretching, biasing force.

As used herein, the terms “elastomer” or “elastomeric” refer to polymeric materials that have properties of stretchability and recovery.

“Stretch” refers to the ability of a material to extend upon application of a biasing force. Percent stretch is the difference between the initial dimension of a material and that same dimension after the material has been stretched or extended following the application of a biasing force. Percent stretch may be expressed as [(stretched length—initial sample length)/initial sample length]×100. For example, if a material having an initial length of one (1) inch is stretched 0.50 inch, that is, to an extended length of 1.50 inches, the material can be said to have a stretch of 50 percent.

“Recover” or “recovery” refers to a contraction of a stretched material upon termination of a biasing force following stretching of the material by application of the biasing force. For example, if a material having a relaxed, unbiased length of one (1) inch is elongated 50 percent by stretching to a length of one and one half (1.5) inches the material would have a stretched length that is 150 percent of its relaxed length. If this exemplary stretched material contracted, that is recovered to a length of one and one tenth (1.1) inches after release of the biasing and stretching force, the material would have recovered 80 percent (0.4 inch) of its elongation.

“Water impermeable,” when used in describing a layer or multi-layer laminate means that water or water vapor will not pass to any appreciable extent through the layer or laminate, under ordinary use conditions, in a direction generally perpendicular to the plane of the layer or laminate at the point of liquid contact.

“Water permeable” refers to any material that is not water impermeable.

These terms may be defined with additional language in the remaining portions of the specification.

### Description

Various problems associated with skin or tissue may diminish the health and/or hygiene of a person or animal. For example, dryness of the skin of the hands, feet, extremities, joints, or other parts of a body is a common problem, especially when skin is exposed to cold and/or dry conditions. This may be especially true for older individuals. Various formulations designed to moisturize skin are often used to address this problem. Some formulations require a substantial amount of time to deliver the anticipated benefit. For many currently available formulations, any resulting skin benefit may last a relatively short period of time after the formulation has been applied. The present invention characterizes an appliance for delivering a formulation or composition to tissue or skin.

One example of such a composition is a skin-care formulation for moisturizing skin. Such formulations typically work through at least one of two mechanisms: occlusivity or humectancy. Skin-care formulations relying on occlusivity form a relatively water-vapor-impermeable film on and/or in a skin surface. This occlusive film results in the accumulation of water underneath the film as the skin undergoes the natural process of trans-epidermal water loss. One advantage of the occlusivity approach includes the ability to provide a skin-moisturization benefit for an extended period of time. Occlusive ingredients are typically hydrophobic in nature and are generally not easily washable, which contribute to their ability to provide long-term moisturization of skin. Formulations with ingredients having occlusive properties (such as peterolatum, waxes, vegetable oils, mineral oil, etc.) are perceived by some as having undesirable aesthetic and/or feel attributes. Occlusive formulations may also lack the ability to provide quick moisturization as such formulations depend on the relatively slow process of water accumulation due to trans-epidermal water loss to deliver the moisturizing benefit.

Humectant formulations have the ability to attract water vapor (moisture) from the atmosphere and bring it to the skin surface, which results in increased skin hydration and alleviation of dryness. This process of attracting moisture is frequently referred to as “hygroscopicity”. Humectant formulations have the advantage of delivering a moisturization benefit and dryness relief within a short period of time. Humectant formulations are typically hydrophilic (as noted above, “water loving”) in nature and generally contain a significant amount of water. Such formulations are generally perceived as having a light, pleasant feel (i.e., light in weight and/or viscosity) on the skin and typically are aesthetically preferred by the user (relative to skin-care compositions that function by occlusivity). Examples of humectant ingredients include glycerin, urea, sodium lactate, polysaccharides, and the like. Unlike occlusive formulations, humectant formulations generally lack the ability to provide moisturization over an extended period of time.

As noted elsewhere in this application, skin care compositions or formulations may cause a variety of problems for elastic materials made of certain types of elastomeric polymers. Styrene-olefin block copolymers (e.g., styrene-ethylene-butylene-styrene tetrablock copolymers), for instance, are oleophilic by nature and tend to swell, soften, and even dissolve in the presence of oleophilic skin care compositions (e.g. oils). Not only does this adversely affect the stretch and recovery properties of the elastic material, but it may also cause the composite to delaminate and thereby destroy the integrity of the product.

Accordingly, in one aspect of the present invention, a skin-care formulation (e.g., a formulation comprising a humectant) is applied or associated with the interior of an appliance such as a glove, sock, sleeve, or patch. The appliance comprises an elastic member, such as an elastomeric film, or individual elastic components, such as elastic strands (e.g., as discussed elsewhere in this application, individual elastic strands may be extruded or formed such that they are spaced apart and substantially parallel, and to these strands may be attached meltblown or other fiber). The elastic member comprises polymers adapted to impart elastomeric qualities to films, strands, fibrous webs, and other such components. Such polymers include, for example, KRATON styrenic block copolymers available from businesses such as Kraton, Kurary, and Dynasol; DEXCO olefinic polymers available from businesses such as Dow Chemical and ExxonMobil; and other such polymers.

The appliance also comprises a shielding member interposed between the elastic member, such as an elastomeric film, a composite of spaced-apart, substantially parallel elastic strands, or some other elastomeric substrate or composite; and any formulation associated or applied to the interior of the appliance. For example, the shielding member can be a film separate from the elastic layer, but made from one or more polymers having a higher crystallinity than polymers used to form the elastic layer. The crystalline polymers serve to impede diffusion or mass transport of one or more ingredients in the formulation that may degrade or negatively interact with elastomeric polymers in the elastic member. Alternatively, the shielding member, in this case a film, can also be composed of one or more polymers having a crystallinity sufficient to impede the mass transport or diffusion of ingredients in the formulation (e.g., oils) that could interact with and degrade elastomeric polymers. Alternatively, the shielding member or layer is selected so that it is impermeable to the formulation.

In some versions of the invention, a single film can be formed by co-extrusion so that the film comprises two or more layers, with each layer composed of different ingredients, and therefore possessing different physical characteristics. So, for example, one layer could be extruded using one or more elastomeric polymers, and a second layer could be extruded with one or more polymers having a crystallinity sufficient to inhibit the passage of certain formulation ingredients (e.g., oils). Or the second layer—again, the shielding member or layer—is impermeable to the formulation as a whole.

The elastic member and shielding member may be sandwiched between two fibrous layers (e.g., nonwoven materials such as polypropylene spunbond materials). Without being bound to a particular theory, we believe an appliance having this configuration and comprising a humectant-type formulation can realize the benefits of both humectancy and occlusivity without their respective disadvantages. A humectant-type formulation on the inside of the appliance provides the aesthetically pleasing feel to the appliance user and delivers the initial quick moisturization benefit to the skin. The occlusive nature of the shielding member/layer, which in the case of a film or a composite comprising a film will typically be impermeable the formulation, helps provide a product that contributes to a longer-term moisturization effect. Furthermore, the fibrous layers give the appliance a cloth-like feel and appearance. Also, undulations in any optional inner fibrous layer (which contacts skin or tissue of the user) helps contain the formulation or composition that is applied to the inner fibrous layer. As discussed elsewhere, these undulations (rugosity) may be effected, in whole or in part, by attaching the fibrous layers to the elastic member at discrete points or locations while the elastic member is in a stretched condition. When the resulting laminate is allowed to contract, the fibrous layers are gathered to enhance or produce undulations in the fibrous layers. Of course, in some versions of the present invention, the elastic member is attached to the shielding member prior to attachment of any optional fibrous layers. If this is the case, then the elastic layer/shielding layer composite is stretched, with the shielding member limiting the elastomeric quality of the composite as a whole.

Note that an appliance made by a method of the present invention may comprise an outer fibrous layer, an inner fibrous layer, both an inner or outer fibrous layer, or no fibrous layer at all. Furthermore, a fibrous layer may be provided as a web separate from either the elastic member or shielding member when making the appliance. Or a fibrous layer can be attached to, for example, the shielding member in one manufacturing step to form a composite web (e.g., by bonding a nonwoven material to a film comprising a crystalline polymer or polymer segment that helps inhibit or impede mass transport or diffusion of the formulation or formulation ingredients through the shielding member), and then supplying the composite web for combining with the elastic member.

### Representative Substrates for Constructing an Appliance of the Present Invention

A substrate used to make an appliance of the present invention will generally have at least two members: an elastic member or layer (such as an elastomeric film, or individual elastic strands); and a shielding member or layer (such as a film employing some amount of a crystalline polymer or polymer segments to help reduce or inhibit the mass transport or diffusion of the formulation and/or certain molecules in the formulation, such as oils, that may interact negatively with one or more elastomeric constituents of the elastic member). Typically the shielding member is impermeable to the formulation; i.e., during the time between manufacture and use, the shielding member inhibits or reduces passage of the formulation through the shielding member so that the elastic member or layer does not suffer significant degradation or loss of its elastomeric qualities, or, where the elastic member is attached to the shielding member using ultrasonic, thermal, adhesive, or other such bonds, the elastic member does not separate or delaminate from the shielding member.

The shielding member is interposed between the elastic member and the formulation or composition employing one or more ingredients that may degrade or negatively interact with elastomeric constituents. As noted above, typically the shielding member also acts as a barrier to the formulation itself, i.e., for an appliance of the present invention adapted to accommodate a body part inserted into the appliance (e.g., inserting a hand into a glove adapted to transfer a formulation to the skin of the hand), the shielding member acts to inhibit transport of the formulation from the interior of the appliance to the exterior of the appliance, where the formulation might then be spread by contact to other surfaces that a user of the appliance touches. As noted above, where the shielding member is a web separate from the elastic member (as compared to, for example, a co-extruded film in which one film layer is attached to a second film layer, with each layer having different ingredients to help effect that layer's function, whether it be to help promote an elastic function or a barrier function), then a maker of the appliance can: (1) if desired, select an elastic member that is porous/liquid-permeable because the shielding member will provide the barrier function of the appliance; (2) select ingredients for the shielding member (helping promote a barrier function in the resulting appliance) and elastic member (helping promote an elastic function in the resulting appliance) that helps promote each member's primary function—without having to balance the two functions as is generally necessary when one web (e.g., one, unstratified, single-layer film) provides both a barrier function and an elastic function).

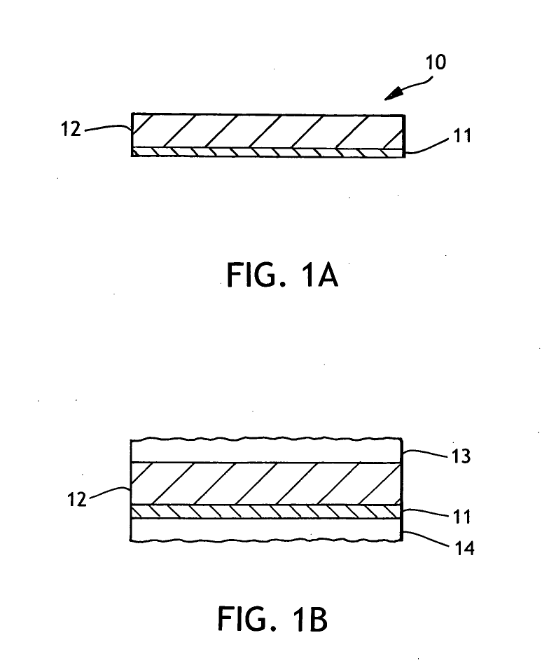

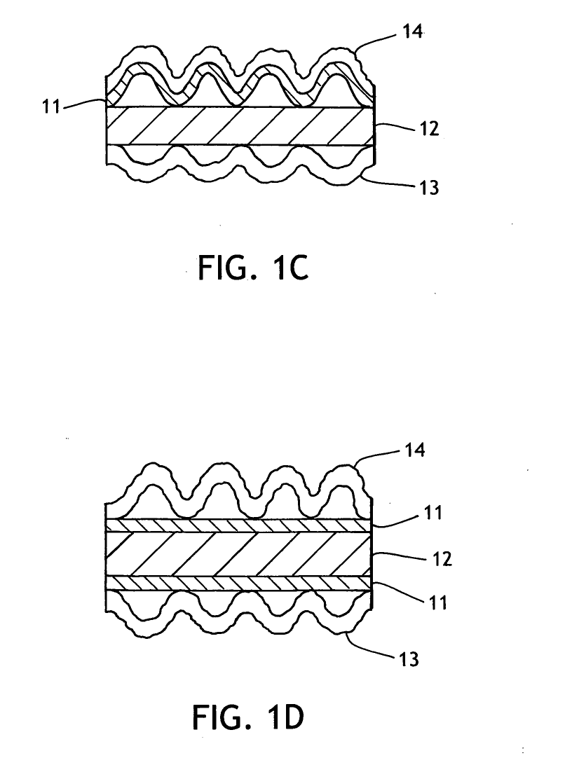

An example of such a substrate 10 is depicted in FIG. 1A, which representatively illustrates an elastic member 12 attached to a shielding member 11 (Note: The drawing is not to scale; the thickness of the shielding member could be the same as, greater than, or less than the thickness of the elastic member). In the example depicted in FIG. 1A, the elastic member 12 is a film. A suitable class of film materials includes a thermoplastic elastomeric polyolefin polymer. These (and other) components can be mixed together, heated, and then extruded into a mono-layer or multi-layer film using any one of a variety of film-producing processes known to those of ordinary skill in the film processing art. Such film-making processes include, for example, cast embossed, chill and flat cast, and blown film processes. Typically the elastic member 12 will be attached to the shielding member 11 using an adhesive, thermal bonding, ultrasonic bonding, or by virtue of the elastic member 12 and shielding member 11 being co-formed together (i.e., by forming a single film comprising one layer serving as an elastic member and one layer serving as a shielding member).

Other additives and ingredients may be added to the elastic member 12 provided they do not significantly interfere with the ability of the elastic member to function in accordance with the teachings of the present invention. Such additives and ingredients can include, for example, antioxidants, stabilizers, and pigments.

In addition to the polyolefin polymer in this representative example, the elastic member 12 can also include a filler. As used herein, a “filler” is meant to include particulates and other forms of materials which can be added to the film polymer extrusion blend and which will not chemically interfere with the extruded film but which are able to be uniformly dispersed throughout the film. Generally, the fillers will be in particulate form and may have a spherical or non-spherical shape with average particle sizes in the range of about 0.1 to about 7 microns. Both organic and inorganic fillers are contemplated to be within the scope of the present invention provided that they do not interfere with the film formation process, or the ability of the film layer to function in accordance with the teachings of the present invention. Examples of suitable fillers include calcium carbonate (CaCO₃), various kinds of clay, silica (SiO₂), alumina, barium carbonate, sodium carbonate, magnesium carbonate, talc, barium sulfate, magnesium sulfate, aluminum sulfate, titanium dioxide (TiO₂), zeolites, cellulose-type powders, kaolin, mica, carbon, calcium oxide, magnesium oxide, aluminum hydroxide, pulp powder, wood powder, cellulose derivatives, chitin and chitin derivatives. A suitable coating, such as, for example, stearic acid, may also be applied to the filler particles.

As mentioned herein, elastic member 12 may be formed using any one of the conventional processes known to those familiar with film formation, if the elastic member is composed of a film. If so, a polyolefin or other polymer and any optional ingredients (e.g., filler) are mixed in and then heated and extruded into a film.

It should be noted that the representative embodiment of an elastic member 12 discussed in the preceding paragraphs may also function as a liquid-impermeable layer, i.e., the elastic member also serves to impede or reduce the amount of liquid, such as water, that will migrate or diffuse through the member. In this way, as discussed elsewhere, the member serves to act as an occlusive material, i.e., the elastic member promotes the retention of, for example, water in a formulation or composition. Typically, when the elastic member is a film, it will also be liquid-impermeable, unless it is perforated, or otherwise altered so that liquid is able to readily pass through it. But, as is discussed elsewhere in the application, the elastic member is not limited to films or other structures that provide a barrier function for the appliance. As noted above, the elastic function of the appliance could be provided by a composite comprising, for example, individual elastic strands attached to meltblown fiber—with the composite being liquid-permeable.

In the embodiment depicted in FIG. 1A, the shielding member 11 is also a film (whether formed separately from, or with [e.g., via co-extrusion], the elastic member 12). The shielding member 11 may be formed from the same kinds of materials identified above for making the elastic member. But the materials for making the shielding member are selected so that the formulation and/or one or more ingredients in a formulation will not pass through the shielding member in sufficient quantities, and at a rate of transport, such that the elastic member degrades; or, alternatively, adsorbs/absorbs sufficient quantities of the formulation or formulation ingredient(s) such that the elastic member, or the appliance as a whole, no longer functions for its intended purpose (e.g., the elastic member loses its ability to stretch and then recover some portion of its original shape; or the elastic member separates from the shielding member, or some other layer attached to the elastic member, whether the elastic member is thermally, adhesively, ultrasonically, or otherwise attached or bonded to the shielding member and/or other layer). This could happen, for example, if oils or oil-based ingredients in the formulation passed through the shielding member 11 and interacted with one or more polymers in the elastic member that help give the elastic member its elastomeric properties. Or if, for example, oils or oil-based ingredients passed through the shielding member and were adsorbed and/or absorbed by the elastic member such that its physical properties were changed to the detriment of the appliance's structural integrity or intended function when employed by a user of the appliance. Accordingly, one or more polymeric ingredients of the shielding member may be selected so that the polymers are of a more highly crystalline nature, thereby helping impede the passage of, for example, the formulation as a whole; or oils or oil-based ingredients; or other ingredients through the shielding member. For example a polypropylene polymer that is composed of between about 10 to about 60 percent by weight crystallinity may be used in a shielding layer. In one version of the invention, a film serving as a shielding layer is composed of between about 2 to about 30 percent by weight of a crystalline polymer. In another version of the invention, a film serving as a shielding layer is composed of between about 5 to about 10 percent by weight of a crystalline polymer. A shielding layer can comprise, for example, mixtures of atactic, syndiotactic, and isotactic polypropylene such that the shielding layer is made with an amount of crystalline polymer segments that differs from the amount of crystalline polymer segments in the elastic layer (or to achieve the above recited amounts of crystallinity). Alternatively, mixtures of atactic, syndiotactic, and/or isotactic polypropylene may be employed in the shielding layer so that the shielding layer is impermeable to the formulation. Other examples of polymers that may be employed in a shielding member/ layer are described in a co-pending U.S. provisional patent application, filed on 27 Sep. 2006, and corresponding to internal docket number 64048978US01. This application is entitled “Elastic Composite Having Barrier Properties,” to Laura Keck, et al., and is hereby incorporated by reference in its entirety in a manner consistent herewith. It should be noted that polymers described in the cited provisional application are selected to balance the barrier function and elastic function, and so may be semi-crystalline in nature. They provide further examples of polymers that may be employed in the shielding member, so long as the shielding member serves its intended function as described elsewhere in this application (i.e., impedes or reduces mass transport of the formulation and/or formulation ingredients through the layer, thereby protecting the elastic layer from significant degradation).

If the substrate depicted in FIG. 1A was used to construct an appliance of the present invention without any additional optional layer, then any formulation to be used with the appliance would be associated with the shielding member 11; i.e., the shielding member would be interposed between elastic member 12 and the formulation (which, for example, could be sprayed, printed, coated, brushed, or otherwise applied or associated with the shielding member).

Another example of an embodiment of the present invention is depicted in FIG. 1B. In essence, FIG. 1B depicts the elastic member and shielding member of FIG. 1A disposed between an inner fibrous layer 14 and an outer fibrous layer 13. The material for the outer fibrous layer 13 may be any material that provides for a cloth-like appearance. The material for the inner fibrous layer 14 may be any material that is fibrous in nature, such as a nonwoven material. The inner fibrous layer could possess an uneven, undulating surface to help contain the formulation or composition applied to, or associated with, the inner fibrous layer 14 and/or the shielding member 11. The rugosity of this inner material can be achieved or enhanced by attaching the inner fibrous layer 14 to the shielding member 11; or to the combination of the shielding member 11 and elastic member 12; at discrete points or locations (e.g., by thermally point bonding the materials together, as is discussed in more detail below) while the shielding member 11 is in a stretched condition (or when the combination of the shielding member 11 and elastic member 12 are in a stretched condition). As noted elsewhere, the shielding member will likely be less elastomeric than the elastic member. Accordingly, other appliance configurations may be used when attaching an inner and/or outer fibrous layer such that the inner and/or outer fibrous layers are gathered in the appliance. Specifically, those configurations may be used where the elastic member can be stretched independently of the shielding member during manufacture of the appliance.

When the elastic member, shielding member, or a combination of a shielding member and elastic member are stretched prior to and/or during intermittent attachment of an inner and/or outer fibrous layer (e.g., by thermal bonding the fibrous layer(s) to the elastic member, shielding member, or some combination thereof at discrete locations), with the resulting composite then allowed relax, the inner fibrous layer 14 is gathered to produce undulations in the inner fibrous layer. Of course, both the inner fibrous layer 14 and the outer fibrous layer 13 are gathered in this way if they are attached to the shielding member and elastic member at discrete points or locations while the member or members are in a stretched condition (and then allowed to relax).

While FIG. 1B depicts both an inner and outer fibrous layer, the shielding member and elastic member could be attached to only a single fibrous layer, whether oriented inward to the tissue or skin of a user of the appliance (i.e., an inner fibrous layer), or toward the exterior of the appliance (i.e., an outer fibrous layer).

As noted elsewhere, any formulation associated with the appliance would be introduced in a way such that the shielding member 11 is interposed between the formulation and the elastic member 12. Accordingly, the formulation would be associated with the inner fibrous layer 14 and/or shielding member 11.

While the description associated with the preceding Figure noted that an inner and/or outer fibrous layer could be gathered (without the Figure actually depicting what one version of a gathered layer looks like), FIG. 1C depicts a substrate having gathered layers and comprising an elastic member and a shielding member. Here elastic member 12 is bonded at discrete locations to an outer fibrous layer 13, and to a composite comprising a shielding member 11 bonded to an inner fibrous layer 14. One way to make the substrate depicted in FIG. 1C is to stretch elastic member 12 prior to its being directed to a nip between two rolls employed to bond the elastic member 12 to the other layers. So, for example, if elastic member 12 is an elastomeric film, then the film is stretched prior to its being directed to the aforementioned nip. To make the embodiment depicted in FIG. 1C, two additional webs are also directed to the same nip: the outer fibrous layer 13 (e.g., a necked polypropylene spunbond material) on one side of the film and the combination of—in this case a laminate of—the shielding member 11 (e.g., a film comprising crystalline ingredients to act as a barrier to a formulation and/or ingredients in the formulation) and an inner fibrous layer 14 (e.g., a necked polypropylene spunbond material). By bonding the outer fibrous layer 13 and the laminate of the shielding member 11 and inner fibrous layer 14 intermittently along the length of the elastic member 12, and then allowing the resulting combination to contract, a substrate like that depicted in FIG. 1C is formed. Both the outer fibrous layer and the laminate are gathered: i.e., at certain locations along the length of the elastic member 12 the outer fibrous layer and the laminate are adjacent, and bonded to, the elastic member. Between these points or regions of adjacency/attachment, the outer fibrous layer and the laminate are not attached to the elastic member, and these unattached portions may appear like “hills” or undulations between the points of attachment. So that the shielding layer 11 of the laminate is interposed between any formulation and the elastic member 12, any formulation would generally be associated with the inner fibrous layer 14.

It should be noted that various patterns may be used when thermally, adhesively, or otherwise bonding two or more layers together. While FIG. 1C provides what is, in effect, a side-view of a cross-section of the substrate, a top-down view would reveal a pattern of the discrete bonding locations by which the two or more layers are attached to one another. For example, a top down view could show discrete bonding areas that resemble the letter “Y”, with unbonded regions around each of these discrete Y-shaped bonded areas.

FIG. 1D depicts another substrate that may be employed in an appliance of the present invention. Elastic member 12 is attached to, or co-extruded with, shielding members 11. The elastic member 12, in this case, is sandwiched between two shielding members—one attached to one surface of the elastic member and the other attached to the opposing surface of the elastic member. One example of such a composite is a co-extruded film comprising 3 layers, with the middle layer serving as the elastic member and comprising one or more elastomeric materials, and the “skin” layers on either side of the middle layer comprising crystalline polymer(s) sufficient to impede or block mass transport of a formulation or one or more formulation ingredients that could chemically or physically degrade the performance of the elastic member (e.g., by chemically interacting with amorphous portions of an elastomeric polymer; by being adsorbed/absorbed by the elastic member such that its physical properties, and the appliance's performance, is degraded; etc.). As in the representative embodiment depicted in FIG. 1C, a formulation associated with said appliance would be associated with, for example, inner fibrous layer 13 so that a shield member was interposed between the formulation and the elastic member.

As noted in the preceding paragraphs describing various representative versions, an inner and/or outer fibrous layer may be attached to the combination of a shielding member and elastic member. The inner and outer fibrous layers may be the same or may be different. The resulting substrate is able to stretch and conform to a hand, foot, extremity, or other body region to which the appliance is applied.

Note too that the elastic member can be porous and/or liquid permeable. For example, the elastic member can be substantially parallel, spaced-apart elastic strands attached to meltblown fiber. As stated elsewhere in this application, because the elastic member need not concurrently act as a barrier layer or shielding member, it can be constructed to effect the elastomeric quality desired in the final appliance without also having to effect a barrier function in the appliance. So the elastic strand/meltblown fiber composite can be permeable to liquid in appliances of the present invention—the shielding member serves to stop or reduce passage of a formulation and/or formulation ingredient(s) to or through the shielding member such that physical or chemical characteristics of the elastic member are degraded or altered, with concurrent degradation of performance of the appliance as a whole (e.g., because oil-like ingredients in the formulation are adsorbed/absorbed by the elastic strands, therefore degrading the elastomeric qualities of the strands and the appliance; and because the formulation and/or formulation ingredient(s), after penetration the shielding member, may be available on an exterior surface of the appliance, thereby contaminating any surface that a user of the appliance contacts when using the appliance).

If a nonwoven material is used to make the optional inner and/or outer fibrous layers, then commercially available thermoplastic polymeric materials can be advantageously employed in making the fibers or filaments from which the outer fibrous layer and inner fibrous layer are formed. As used herein, the term “polymer” shall include, but is not limited to, homopolymer, copolymers, such as, for example, block, graft, random and alternating copolymers, terpolymers, etc., and blends and modifications thereof. Moreover, unless otherwise specifically limited, the term “polymer” shall include all possible geometric configurations of the material, including, without limitation, isotactic, syndiotactic, random and atactic symmetries. As used herein, the terms “thermoplastic polymer” or “thermoplastic polymeric material” refer to a long-chain polymer that softens when exposed to heat and returns to the solid state when cooled to ambient temperature. Exemplary thermoplastic materials include, without limitation, polyvinyl chlorides, polyesters, polyamides, polyfluorocarbons, polyolefins, polyurethanes, polystyrenes, polyvinyl alcohols, caprolactams, and copolymers of the foregoing.

Nonwoven webs that can be employed as the optional fibrous layers of the present invention can be formed by a variety of known forming processes, including spunbonding, airlaying, meltblowing, or bonded carded web formation processes. Spunbond nonwoven webs are made from melt-spun filaments. As used herein, the term “meltspun filaments” refers to small diameter fibers and/or filaments which are formed by extruding a molten thermoplastic material as filaments from a plurality of fine, usually circular, capillaries of a spinneret with the diameter of the extruded filaments then being rapidly reduced, for example, by non-eductive or eductive fluid drawing or other well known spunbonding mechanisms. Lastly, the melt-spun filaments are deposited in a substantially random manner onto a moving carrier belt or the like to form a web of substantially continuous and randomly arranged, melt-spun filaments. Spunbond filaments generally are not tacky when they are deposited onto the collecting surface. The production of spunbond nonwoven webs is described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,538 to Peterson, and U.S. Pat. No. 3,542,615 to Dobo et al., all of which are incorporated herein by reference. The melt-spun filaments formed by the spunbond process are generally continuous and have average diameters larger than 7 microns based upon at least 5 measurements, and more particularly, between about 10 and 100 microns. Another frequently used expression of fiber or filament diameter is denier, which is defined as grams per 9000 meters of a fiber or filament.

Spunbond webs generally are stabilized or consolidated (pre-bonded) in some manner immediately as they are produced in order to give the web sufficient integrity and strength to withstand the rigors of further processing into a finished product. This pre-bonding step may be accomplished through the use of an adhesive applied to the filaments as a liquid or powder which may be heat activated, or more commonly, by compaction rolls. As used herein, the term “compaction rolls” means a set of rollers above and below the nonwoven web used to compact the web as a way of treating a just produced, melt-spun filament, particularly spunbond, web, in order to give the web sufficient integrity for further processing, but not the relatively strong bonding of later applied, secondary bonding processes, such as through-air bonding, thermal bonding, ultrasonic bonding and the like. Compaction rolls slightly squeeze the web in order to increase its self-adherence and thereby its integrity.

An exemplary secondary bonding process utilizes a patterned roller arrangement for thermally bonding the spunbond web. The roller arrangement typically includes a patterned bonding roll and a smooth anvil roll which together define a thermal patterning bonding nip. Alternatively, the anvil roll may also bear a bonding pattern on its outer surface. The pattern roll is heated to a suitable bonding temperature by conventional heating means and is rotated by conventional drive means, so that when the spunbond web passes through the nip, a series of thermal pattern bonds is formed. Nip pressure within the nip should be sufficient to achieve the desired degree of bonding of the web, given the line speed, bonding temperature and materials forming the web. Percent bond areas within the range of from about 10 percent to about 20 percent are typical for such spunbond webs.

If a film, the elastic member can be formed of any film to yield a substrate/appliance having the performance characteristics and features described herein. A suitable class of film materials includes a thermoplastic elastomeric polyolefin polymer, including, for example, polypropylene. Suitable propylene polymers are commercially available under the designations VISTAMAXX™ from ExxonMobil Chemical Co. of Houston, Tex.; FINA™ (e.g., 8573) from Atofina Chemicals of Feluy, Belgium; TAFMER™ available from Mitsui Petrochemical Industries; and VERSIFY™ available from Dow Chemical Co. of Midland, Mich. Other examples of suitable propylene polymers are described in U.S. Pat. Nos. 7,105,609 to Datta, et al.; 6,500,563 to Datta. et al.; 5,539,056 to Yang, et al.; and 5,596,052 to Resconi, et al., which are incorporated herein in their entirety by reference thereto for all purposes.

Of course, other thermoplastic polymers may also be used to form the elastic film so long as they do not adversely affect the elastic properties of the film (or as discussed elsewhere, e.g., strands, or composites). For example, the elastic film may contain other polyolefins, elastomeric polyesters, polyurethanes, polyamides, block copolymers, and so forth. For example, polyethylene may be employed in some embodiments of the present invention. The density of the polyethylene may vary depending on the type of polymer employed, but generally ranges from 0.85 to 0.96 grams per cubic centimeter (“g/cm³”). Polyethylene “plastomers”, for instance, may have a density in the range of from 0.85 to 0.91 g/cm³. Likewise, “linear low density polyethylene” (“LLDPE”) may have a density in the range of from 0.91 to 0.940 g/cm³; “low density polyethylene” (“LDPE”) may have a density in the range of from 0.910 to 0.940 g/cm³; and “high density polyethylene” (“HDPE”) may have density in the range of from 0.940 to 0.960 g/cm³.

Besides polymers, the elastic film of the present invention may also contain other additives as is known in the art, such as melt stabilizers, processing stabilizers, heat stabilizers, light stabilizers, antioxidants, heat aging stabilizers, whitening agents, antiblocking agents, bonding agents, tackifiers, viscosity modifiers, etc. Suitable viscosity modifiers may include, for instance, polyethylene wax (e.g., EPOLENE™ C-10 from Eastman Chemical). Phosphite stabilizers (e.g., IRGAFOS available from Ciba Specialty Chemicals of Terrytown, N.Y. and DOVERPHOS available from Dover Chemical Corp. of Dover, Ohio) are exemplary melt stabilizers. In addition, hindered amine stabilizers (e.g., CHIMASSORB available from Ciba Specialty Chemicals) are exemplary heat and light stabilizers. Further, hindered phenols are commonly used as an antioxidant in the production of films. Some suitable hindered phenols include those available from Ciba Specialty Chemicals of under the trade name “Irganox®”, such as Irganox® 1076, 1010, or E 201. Moreover, bonding agents may also be added to the film to facilitate bonding of the film to additional materials (e.g., nonwoven web). When employed, such additives (e.g., tackifier, antioxidant, stabilizer, etc.) may each be present in an amount from about 0.001 wt. % to about 25 wt. %, in some embodiments, from about 0.005 wt. % to about 20 wt. %, and in some embodiments, from 0.01 wt. % to about 15 wt. % of the film.

These (and other) components can be mixed together, heated and then extruded into a mono-layer or multi-layer film using any one of a variety of film-producing processes known to those of ordinary skill in the film processing art. Such film-making processes include, for example, cast embossed, chill and flat cast, and blown film processes. If the elastic member is attached to an outer fibrous layer, then the elastic member will typically be attached to the outer fibrous layer by thermally bonding the layers together at discrete points (see, e.g., discussion in preceding paragraph as well as U.S. Pat. No. 6,037,281, entitled “Cloth-Like, Liquid-Impervious, Breathable Composite Barrier Fabric,” to Mathis, et al.). As noted above, the optional fibrous layer may be bonded or attached to the elastic member at discrete locations while the elastic member is in a stretched condition, thereby producing undulations when the resulting laminate is in a relaxed condition. Other known means for bonding and laminating the elastic member to a fibrous layer may be used, provided the resulting substrate/appliance has the required properties described herein. For example, the layers may be adhesively bonded or ultrasonically bonded to one another.

The preceding paragraphs describe some examples of a substrate that may be used in an appliance of the present invention. For additional examples, see, e.g., U.S. Pat. No. 6,037,281, entitled “Cloth-Like, Liquid-Impervious, Breathable Composite Barrier Fabric,” to Mathis, et al.; U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT Application WO 01/88245 in the name of Welch et al.; all of which are incorporated herein by reference in a manner consistent herewith.

### Representative Appliance Configurations Made in Accordance with the Present Invention

One or more substrates, such as those described above, may be configured into the form of a glove, mitten, sock, sleeve, patch, or other article designed to be fitted to a part of the body. Generally the appliance will be made by cutting a substrate into appropriate pieces such that the pieces, when attached to one another, form an appliance having an interior volume into which a portion of a body may be inserted. But, as noted above, the appliance may be configured in the form of a patch. Typically a formulation will be associated with the appliance during manufacture so that the appliance is ready to use. In some versions of the present invention, however, a formulation is not pre-applied to the appliance, allowing a user to choose and apply a formulation or composition to his or her skin, and then don or affix an appliance of the present invention to the skin or tissue to which the formulation or composition was applied.

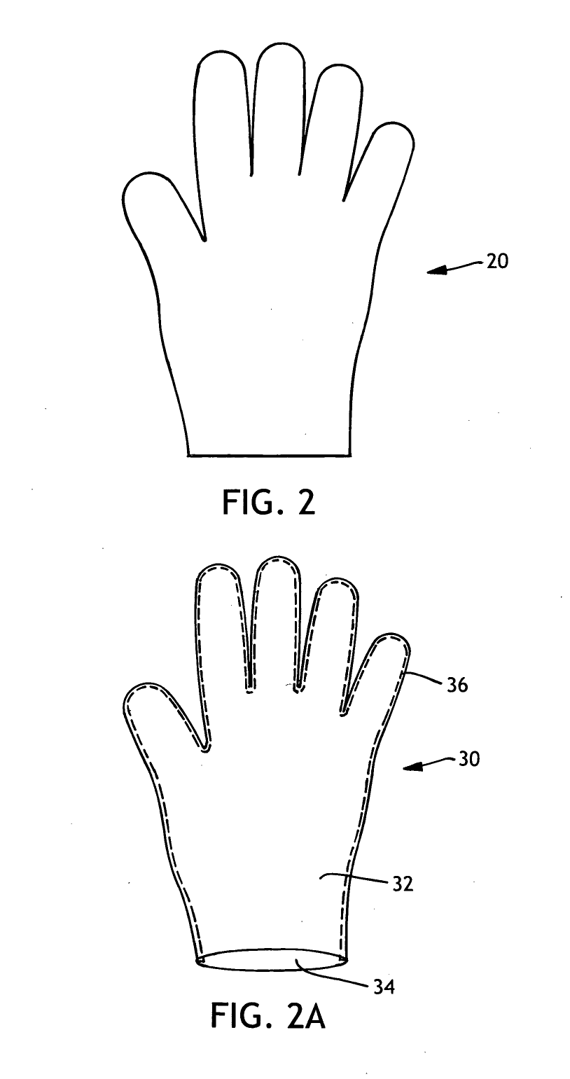

FIG. 2 representatively depicts a substrate 20 cut so that the piece (or substrate) defines a perimeter in the shape of a human hand. FIG. 2A representatively depicts an appliance 30 comprising a first piece (or substrate) 32 attached to a second piece (or substrate) 34 at a location proximate to the perimeters of these two substrates. In this representative illustration, the two substrates are attached to one another mechanically by sewing the pieces together at a location proximate to the perimeters of the two substrates. The resulting appliance was then inverted so that the seam 36 formed by sewing the substrates together is on the interior of the appliance. Of course the finished appliance need not be inverted; the seam can remain on the exterior of the appliance. Note, too, that the individual pieces need not be joined in a way that produces a seam. The edges of the individual pieces may be butted together, and then, for example, joined and/or welded together using a solvent. Alternatively, the individual pieces may be butted together, and another material, such as an adhesive or an adhesive tape, used to join the pieces together. Or the pieces may be thermally bonded or ultrasonically bonded. Furthermore, any glove-like appliance may be formed such that the appliance resembles a bicycle glove, or some portion thereof (i.e., one or more end portions of the individual thumb-like and/or finger-like projections of the glove-like appliance are absent, so that a person may more easily manipulate objects while wearing the appliance because some portion of one or more fingers and/or the thumb is exposed [and at the same time treat skin, for example, at joints, the back of the hand, the palm, or some combination thereof]). Alternatively, a sock may be formed such that a portion proximate to the heel, the toe(s), or some other portion of a user's foot is exposed.

Individual pieces (or substrates) may be cut into a variety of shapes and sizes. Rather than the glove depicted in FIGS. 2 and 2A, the pieces may be cut so that the resulting appliance is in the shape of a tube, sleeve, mitten, sock, or the like. Any shape is possible, so long as the resulting appliance defines an interior volume (for those versions of the invention in which the appliance defines an interior volume) into which a user may insert a portion of his or her body (e.g., a finger, toe, hand, foot, wrist, forearm, etc.) such that a composition applied to, or associated with, the interior surface of the appliance may be transferred to skin or tissue in contact with the interior surface of the appliance. As noted elsewhere, however, in some versions of the invention the appliance is a patch that is applied or affixed to skin (e.g., a patch comprising a body adhesive proximate to the perimeter of the patch, thereby allowing the patch to be releasably affixed to the skin). Furthermore, as stated elsewhere, in some versions of the invention the formulation or composition is applied separately to the skin, followed by a user employing an appliance of the present invention.

The individual substrates or pieces need not be sewn together. The individual pieces or substrates may also be joined ultrasonically, thermally, adhesively, cohesively, using tape, by fusing the materials together (e.g., by using an appropriate solvent), by welding the materials together, or by other approaches. So long as the individual pieces or substrates remain attached or connected during normal use of the appliance, and attachment or connection is such that any composition or formulation on the interior surface of the appliance is contained within the appliance (i.e., there is minimal or no leakage of the formulation or composition), any connection or attachment may be used.

Alternatively, a substrate could be prepared in the form of a rectangle, oval, or other shape (e.g., as for a patch). An adhesive capable of adhering to skin could then be applied to all or part of the perimeter of the shape such that the appliance could be releasably adhered to skin. Any composition to be transferred to skin could then be coated or deposited on the surface of the appliance that will contact skin or tissue.

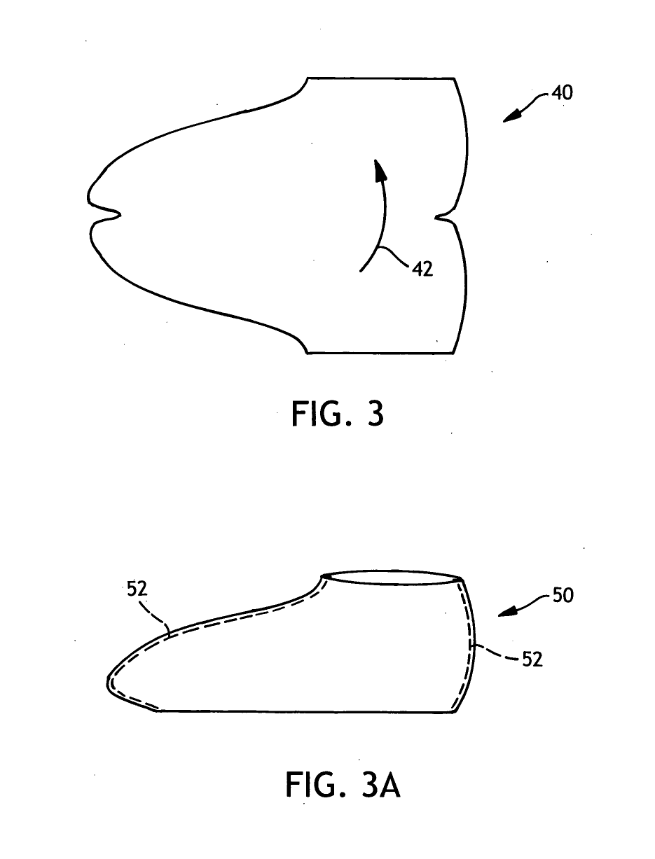

Note, too, that an appliance defining some interior volume may be formed from a single piece of substrate. FIG. 3 representatively illustrates a substrate 40 that has been cut in a way that a foot-shaped appliance may be formed by folding the substrate back on itself (as shown by arrow 42; the bottom half of the shape is folded upward, and on top of, the top half of the shape). FIG. 3A representatively illustrates such a foot-shape appliance 50 and the resulting seams 52 formed when the substrate 40 (from FIG. 3) is folded back, and attached to, itself. In this representative embodiment, the foot-shape appliance was inverted after the substrate was attached to itself so that the seams were on the inside of the appliance. As with two (or more) pieces that may be joined together to form an appliance of the present invention, a single piece may be joined to itself using any of the approaches discussed above (e.g., to form the foot appliance, a sleeve, etc.).

### Representative Processes for Making Aforementioned Substrates

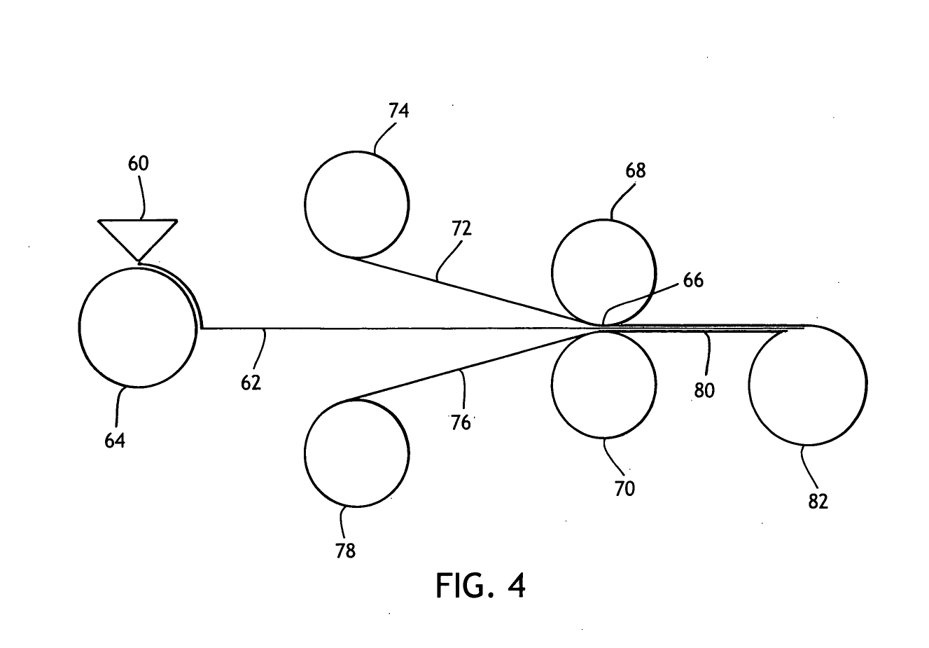

In one version of a process for making a substrate to be employed in an appliance of the present invention, two or more individual webs are directed to a nip between two rolls where the webs are bonded or joined to one another. So, for example in one version of a process as depicted in FIG. 4, a film die 60 is used to cast a film 62 on a chill roll 64, with the film layer then directed to a nip 66 between a pattern roll 68 and an anvil roll 70. While not shown, the film could include multiple individual layers that are extruded onto one another, or which are formed individually and then attached to one another. For example, for the representative version of a process depicted in FIG. 4, the film could include an elastic member—i.e., a center layer of the film comprising an elastomeric polymer—sandwiched between two shielding members, with one shielding member attached to one face of the central elastic layer, and the second shielding member attached to the opposing face of the central elastic layer.

While a film comprising an elastic member and a shielding member could be used as is, so long as the shielding member was interposed between a formulation and the elastic member, the process version depicted in FIG. 4 shows two additional webs being directed to nip 66. One fibrous web 72; e.g. a spunbond nonwoven material, a necked spunbond nonwoven material, or some other nonwoven material; is directed to the nip from unwind stand 74. And a second fibrous web 76 is directed to the nip from unwind stand 78. Note that the film could also be attached to a single fibrous layer.

As noted elsewhere in this Description, the film 62—which in the depicted example of a process includes both an elastic member and a shielding member—can be elongated prior to attachment of any optional fibrous webs. Because in this case the shielding members—which will tend to have more crystalline polymer segments and therefore be less elastomeric—are attached/co-formed with the elastic member, the ability of the film to stretch is lessened. Nevertheless, the film 62 may be stretched prior to attachment at nip 66 to fibrous layers 72 and 76. Typically the film would be stretched by rotating rolls 68 and 70 at a higher rotational speed than the rotational speed of roll 64 on which the film is being cast. The rotational speed of unwind stands 74 and 78 can match the rotational speed of rolls 68 and 70—i.e., the fibrous webs are not elongated when attached to the film 62. Of course, if desired, the fibrous webs could also be elongated prior to attachment to the film 62.

Typically the webs 72 and 76 are bonded to the film 62 by inputting energy in some form (e.g., heat, ultrasonics, and the like), with or without an adhesive being applied. While not shown, an adhesive could be coated, sprayed, meltblown, or otherwise applied to one or more surfaces associated with the film and/or webs to be joined. Furthermore, it should be noted that the composition of the shielding members may be selected to facilitate joining to any optional film layer. For example, the shielding members, as mentioned earlier, could comprise a blend of atactic, syndiotactic, random, and/or isotactic polypropylene. If so, and one or more polymeric ingredients of the shielding member are similar to one or more polymeric ingredients in one or both fibrous webs (i.e., polypropylene in some form is used in both a shielding member and a fibrous web to which the shielding member will be attached), then bonding or attachment could be facilitated or optimized.

The resulting substrate 80 comprising the aforementioned webs is then allowed to retract, typically resulting in a substrate with gathered fibrous webs. The substrate 80 can be wound up on a winder 82 for further converting/processing into appliances of the present invention. Or the substrate 80 could, rather than be directed to a winder, be processed further using, for example, die cutting, slitting, bonding, and/or other various converting operations known to those of skill in the art of manufacturing articles from webs and other components.

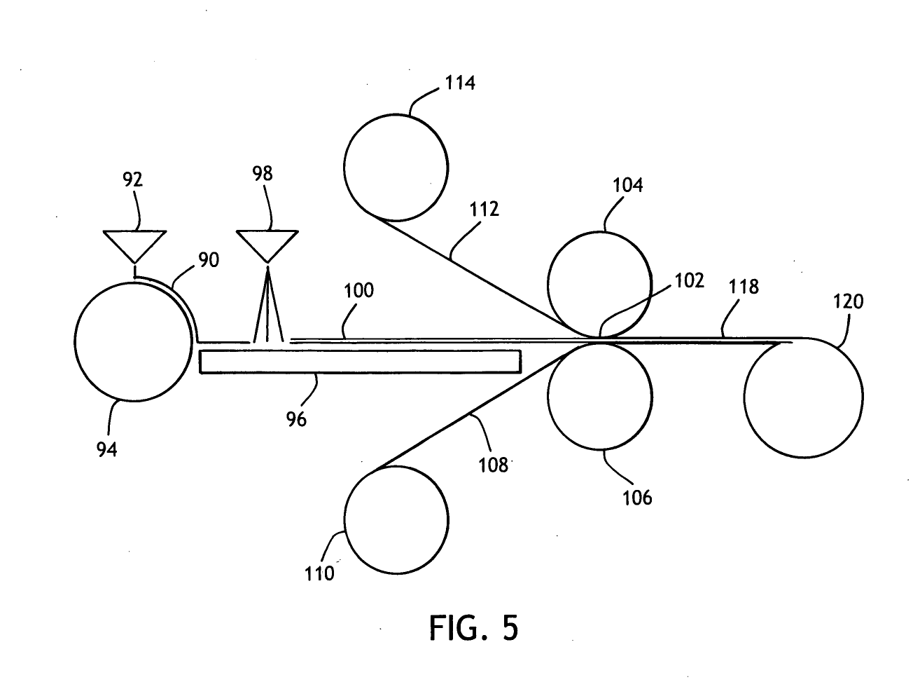

Another version of a process of the present invention is depicted in FIG. 5. In this particular version, spaced-apart, substantially parallel elastic strands 90 are extruded from a filament die 92 onto a chill roll 94. These strands are then directed onto a conveyor 96. A meltblown die 98 is then used to form and direct meltblown fiber onto the already-formed elastic strands, resulting in a composite 100 of spaced-apart, substantially parallel elastic strands attached to meltblown fiber. Note that a meltblown fiber web could be formed before, after, or substantially concurrently to the formation of the elastic material.

The composite 100 is then directed to a nip 102 between a pattern roll 104 and an anvil roll 106. If desired, the rotational speed of the pattern roll and anvil roll relative to the linear velocity of the composite 100 may be such that the composite is stretched as it enters the nip, and prior to the attachment of any optional webs to one or both surfaces of the composite.

As stated elsewhere in the Description or Summary, by decoupling a barrier function of the appliance from an elastic function of the appliance, a product designer has increased flexibility in choosing the chemical ingredients and/or physical structure of the web or substrate that primarily serves to provide that function of the appliance. Here the elastic function of an appliance is to be primarily supplied by a composite that may be porous and liquid-permeable. This is possible because other webs or components of the appliance will primarily supply a barrier function of the appliance. Furthermore, the product designer can choose polymeric or other ingredients of the elastic strand or material, as well as, in this case, the meltblown fiber, without being concerned about how a formulation, or ingredients in a formulation, may degrade the physical and/or chemical characteristics of these ingredients (or the composite comprising the elastic fiber/material and meltblown fiber). This is because a separate layer—a shielding member—will act to help keep the formulation and/or formulation ingredients away from the elastic member.

For the representative process version depicted in FIG. 5, an outer fibrous web 108 (e.g., a polypropylene spunbond material; a necked polypropylene spunbond material; or other such webs) is directed to nip 102 from an unwind stand 110. In the particular version depicted in FIG. 5, one surface of the fibrous web—the surface that will be attached to the composite—is oriented toward the spaced-apart, substantially parallel, elastic strands of the composite, i.e., the fibrous web contacts the bottom surface of the composite, and because in this representative version the elastic strands are formed first, transferred, and rest on a conveyor surface, with meltblown fiber then deposited on top of the elastic strands, the fibrous layer is adjacent to this bottom surface of the composite when attached to the composite.

A laminate 112 of a shielding member, in this case a film comprising a crystalline polymer sufficient to stop or reduce passage of a formulation and/or formulation ingredients through said film, and a fibrous web, is also directed to nip 102 from an unwind stand 114. In the process version depicted in FIG. 5, the shielding member, in this case a film, is oriented such that the film will contact at least some portion of the meltblown fiber of the composite. The fibrous layer of the laminate does not, i.e., the film portion of the laminate contacts the top surface of the composite, and because in this representative version the elastic strands are formed first, transferred, and rest on a conveyor surface, with meltblown fiber then deposited on top of the elastic strands, the film portion of the laminate is adjacent to this top surface of the composite when attached to the composite.

Typically the web 108 and laminate 112 are bonded to the composite 100 by inputting energy in some form (e.g., heat, ultrasonics, and the like), with or without an adhesive being applied. While not shown, an adhesive could be coated, sprayed, meltblown, or otherwise applied to one or more surfaces associated with fibrous web, composite, and/or laminate.

If the composite 100 is stretched prior to its being directed to nip 102, and the fibrous web 108 and laminate 112 are not stretched (or not stretched to the same degree as the composite), then, by intermittently bonding the fibrous web 108 and the laminate 112 to the composite 100, a substrate 118 is produced having a gathered fibrous web and a gathered laminate, i.e., both the fibrous web and the laminate are adjacent to the composite at points of attachment, but between these points of attachment the fibrous web and laminate are unattached to the composite, typically resulting in an undulating surface.

In the process version depicted in FIG. 5, the substrate is then directed to a wind-up reel 120. The wound-up roll of substrate can then be transported to other locations, or used in the same location, for further converting (e.g., cutting the substrate into strips or pieces adapted for further conversion into specific appliances, e.g., a patch, sleeve, sock, glove, etc.). But, as is discussed elsewhere in the Description, the substrate 118 could be directed to additional unit operations (not shown) rather than to a wind-up reel.

The appliance employing the substrate typically would be used such that the shielding member is interposed between a formulation and the elastic member. So, for example, if a formulation was applied to the substrate 118 before or after converting into a specific appliance such as a sock or glove, the formulation would be typically be associated with the fibrous layer of the laminate (i.e., the fibrous layer adjacent to the film comprising crystalline polymer(s) or polymer portions sufficient to inhibit or stop the passage of the formulation and/or formulation ingredient(s) through the film).

### Representative Formulations or Compositions for Use with an Appliance of the Present Invention

Formulations or compositions that may be used with an appliance of the present invention include emulsifiers, surfactants, viscosity modifiers, natural moisturizing factors, antimicrobial actives, pH modifiers, enzyme inhibitors/inactivators, suspending agents, pigments, dyes, colorants, buffers, perfumes, antibacterial actives, antifungal actives, pharmaceutical actives, film formers, deodorants, opacifiers, astringents, solvents, organic acids, preservatives, drugs, vitamins, aloe vera, and the like.

In some versions of the invention, a clinically beneficial additive of the formulation or composition may either interact directly with epithelial tissue at the cellular level to provide a benefit to the skin, or alternatively, may interact with components at or near the skin surface in order to provide a benefit to the skin.

In one embodiment, the clinically beneficial additive may be an emollient, which is herein defined as an agent that helps restore dry skin to a more normal moisture balance. Emollients act on the skin by supplying fats and oils that blend in with skin, making it pliable, repairing some of the cracks and fissures in the stratum corneum, and forming a protective film that traps water in the skin (i.e., are adapted to occlude water). Emollients that may be suitable for use with the present invention include beeswax, butyl stearate, cermides, cetyl palmitate, eucerit, isohexadecane, isopropyl palmitate, isopropyl myristate, mink oil, mineral oil, nut oil, oleyl alcohol, petroleum jelly or petrolatum, glyceral stearate, avocado oil, jojoba oil, lanolin (or woolwax), lanolin derivatives such as lanolin alcohol, retinyl palmitate (a vitamin A derivative), cetearyl alcohol, squalane, squalene, stearic acid, stearyl alcohol, myristal myristate, certain hydrogel emollients, various lipids, decyl oleate and castor oil.

A preferred clinically beneficial additive is a humectant, which is herein defined to be an agent that supplies the skin with water by attracting moisture from the air and holding it on the skin. Humectants that may be suitable for use with the present invention include alanine, glycerin, PEG, propylene glycol, butylenes glycol, glycerin (glycol), hyaluronic acid, Natural Moisturizing Factor (a mixture of amino acids and salts that are among the skin's natural humectants), saccharide isomerate, sodium lactate, sorbitol, urea, and sodium PCA.

Other clinically beneficial agents that may be suitable for use with the present invention include antioxidants, a unique group of substances that protect a body or other objects from oxidizing. Antioxidants prevent or slow the oxidation process, thereby protecting the skin from premature aging. Exemplary antioxidants for use in the present invention include ascorbic acid ester, vitamin C (ascorbic acid), vitamin E (lecithin), Alpha-Glycosyl Rutin (AGR, or Alpha Flavon, a plant-derived antioxidant), and coenzyme Q10 (also known as ubiquinone).

Other clinically beneficial agents which may be delivered to the skin during use include chelating agents, such as EDTA; absorptive/neutralizing agents, such as kaolin, hectorite, smectite, or bentonite; other vitamins and vitamin sources and derivatives, such as panthenol, retinyl palmitate, tocopherol, and tocopherol acetate; and anti-irritants such as chitin and chitosan.

Additional examples of beneficial agents include skin conditioners, which are herein defined as agents that may help the skin retain moisture, improve softness, or improve texture. Skin conditioners include, for example, amino acids, including alanine, serine, and glycine; allantoin, keratin, and methyl glucose dioleate; alpha-hydroxy acids, including lactic acid and glycolic acid, which act by loosening dead skin cells from the skin's surface; moisturizers (agents that add or hold water in dry skin), including echinacea (an extract of the coneflower plant), shea butter, and certain silicones, including cyclomethicon, dimethicone, and simethicone.

Other examples of beneficial botanical agents, extracts, or other materials that may be suitable for use with the present invention include almonds, chamomile extracts such as bisabolol (believed to relieve irritation, swelling and itching in the skin), elder flowers, honey, safflower oil, and elastin (safflower oil and elastin are believed to aid in retaining skin elasticity).

In addition to one or more clinically beneficial additives, other additives may be included in the formulation or composition. For example, a silicone polymer may be included to improve the slip characteristics of the elastomeric article. Possible silicone polymers include reactive silicones, non-reactive silicones, or a mixture of reactive and non-reactive silicones. Suitable silicones may include, for example, aminosilicones, polyether-modified amino silicones, amino-substituted siloxanes having terminal hydroxy groups, epoxy silicones, quaternary silicones, dimethicone, silicone polyethers, polyether epoxy silicones, silanol fluids, polysiloxy linoleyl pyrrolidone phospholipids, and combinations of possible silicones.

Other additives may be included, for example, glucose derived polymers, or mixtures containing glucose derived polymers (e.g., lauryl glucoside available from Cospha under the trade designation Planteran PS 400), silica, silica dispersions, wetting agents, and preservatives (i.e., parabens, such as methylparaben and propylparaben). In one embodiment, the personal-care composition may include emulsion stabilizers. Exemplary emulsion stabilizers include aluminum stearate, magnesium sulfate, hydrated silica, and ozokerite.

In another embodiment a beneficial agent may be held in the formulation or composition in liposomes. A liposome is a vehicle for delivering agents to the skin. More specifically, a liposome is a microscopic sphere formed from a fatty compound, a lipid, surrounding a water-based agent, such as a moisturizer or an emollient. When the liposome is rubbed into the skin, it releases the agent throughout the stratum corneum.

In another embodiment, the beneficial agent may be present in the carrier in the form of a microencapsulant. A microencapsulant is a sphere of an emollient surrounded by a gelatin membrane that prevents the emollient from reacting with other ingredients in the coating composition and helps distribute the emollient more evenly when pressure is applied and the membrane is broken. The process of forming these beads is called microencapsulation and is generally known in the art.

The formulation or composition of the present invention may be applied to the appliance as an aqueous solution, a dispersion, or an emulsion. In one embodiment, an aqueous composition may be formed including from about 4.5% to about 6% by weight of a humectant. In other embodiments, the humectant may be present at 30% or more by weight. In some other embodiments, the humectant may be present at about 10 to about 20% by weight. In still other embodiments, the humectant is present at about 5 to about 40% by weight. This composition may then be applied to the interior surface of an appliance of the present invention.

In one embodiment, the personal-care composition may be applied as an emulsion. In one embodiment, the formulation or composition may be applied to the surface of the appliance as a micro-emulsion. A micro-emulsion is a particularly fine-particle emulsion that can be applied in a spray form. The particle size of a micro-emulsion is generally less than about one micron, whereas traditional emulsions demonstrate particle sizes of greater than about 50 microns.

The components of a formulation or composition may be applied or associated in combination or separately to the surface of the appliance. For example, a 100% humectant composition may be applied, followed by another 100% beneficial additive composition, such that the two (or more) separate applications together form the coating of the appliance. In such a manner, layers of additives may be built up on the surface of the appliance.

The coating may be deposited on the interior surface of the appliances by any suitable method. For example, the appliances may be dipped in the coating. In an alternative embodiment, the appliances may be tumbled in the coating. In various embodiments, the coating may be applied to the surface of the appliance through dipping, immersion, spraying, patting, printing, brushing, or any other application method known in the art.

In one embodiment, the coating may be sprayed onto a skin-contacting surface of the appliance. For instance, appliances may be placed in a tumbling apparatus while a solution of the coating is sprayed on the gloves. In one embodiment, the spraying process may be repeated. For instance, the spraying process may be repeated up to about twenty times to coat the inner surface of the gloves. In one embodiment, the spraying process may be carried out for a total of between about ten and about twenty times.

In some versions of the present invention, a formulation is associated with an appliance at discrete locations on the appliance. So, for example, in a glove appliance, a formulation could be applied only to those portions of the appliance adapted to contact: the back of the hand; the knuckles of the hand; the joints of the fingers; other such locations; and various combinations thereof. Furthermore, formulations having different ingredients could be associated different locations on the appliance. Any such combination is possible, so long as the shielding member of the appliance is interposed between the elastic member and any formulation or composition that will substantially degrade the elastic member.

### Representative Business Arrangements by which a Business Entity can have an Appliance Designed and/or Assembled by Other Business Entities

One business entity (e.g., a seller—such as a person, partnership, corporation, or the like) may work with one or more other business entities under a variety of business arrangements such that other business entities participate in the assembly of an appliance in accordance with the method of the present invention. Often a business entity works with one or more business entities in other countries where the prevailing wage structure offers lower overhead and/or direct labor and/or other costs associated with making a product. Alternatively, one business entity works with another so each is able to specialize in activities viewed as that entity's core competency. Thus one company having knowledge about consumers' preferences for products in a given product category, e.g., products addressing skin care, may work with another business entity that is good at making products, or components of such products. Accordingly, one business entity may work with one or more other business entities under a business arrangement such that said one or more other business entities carry out a method of the present invention.

In one example of a business arrangement, a seller in the United States sells an appliance that is assembled by another business entity (e.g., an entity—e.g., a contract manufacturer—in China with manufacturing expertise in the area of making products comprising nonwoven and/or film substrates; and/or in the area of forming such films and nonwoven materials) in accordance with the process of the present invention. Frequently the seller will provide some or all of the specifications for the product based on the seller's knowledge of the United States market for that specific product (e.g., the price that a U.S. consumer is typically willing to pay for a product of that type; some or all of the product specifications such that the product provides the anticipated benefit(s) sought by the consumer when purchasing the product; etc.). In some business arrangements, the seller and overseas manufacturer may collaborate to optimize product performance and cost of goods sold for the intended marketing niche.

In another example of a business arrangement, a seller in the United States sells an appliance that is assembled by business entities in another country (e.g., China, Malaysia, South Korea, India, Brazil, etc.) in accordance with the process of the present invention, with each business entity undertaking one or more activities under the business arrangement (e.g., formulate a composition that is associated with the appliance; form a substrate comprising a shielding layer and elastic layer; converting said substrate into an appliance adapted to fit or be releasably engaged to some part of the body of a user; etc.).

Such business arrangements will typically implicate negotiated agreements, most of which are put into written form and signed. But arrangements can also include oral understandings between business entities. Frequently a business arrangement is made well in advance of one entity selling a product that is designed and/or made and/or marketed through the efforts of more than one business entity, perhaps one, two, or more years prior to the business entities undertaking activities to design, make, and sell the product (e.g., an appliance of the present invention).

### EXAMPLES

### Example 1

### Representative Example of a Personal-Care Composition

An exemplary personal-care composition was prepared having the ingredients/components and proportions identified below:

| Component | Weight % | Supplier | Address |
|---|---|---|---|
| Water | 66.1 | N/A | N/A |
| Emulgade CM | 20.0 | Cognis | 300 Brookside Ave, |
| | | | Ambler, PA 19002 |
| Glycerin | 4.0 | Glenn Corp. | 4886 Highway 61 N, |
| (99.7% USP) | | | St. Paul, MN 55110 |
| Hispagel 200 | 4.8 | Cognis | 300 Brookside Ave, |
| | | | Ambler, PA 19002 |
| Sepigel 501 | 3.2 | Seppic | 30, Two Bridges |
| | | | Road, Fairfield, New |
| | | | Jersey 07004 |
| Mackernium-007 | 1.0 | McIntyre Group | 24601 Governors |
| | | | Highway, University |
| | | | Park, IL 60466 |
| Paragon III | 0.5 | McIntyre Group | 24601 Governors |
| | | | Highway, University |
| | | | Park, IL 60466 |
| Fragrance | 0.05 | | |
| Tween 40 | 0.05 | Uniquema | 76 East 24^{th} St, |
| | | | Paterson, NJ 07544 |
| Sodium citrate | 0.3 | Sigma-Aldrich | 3050 Spruce Street, |
| 20% | | | St. Louis, MO 63103 |

The recited proportions of water, glycerin, Emulgade CM, Mackernium-007, and Paragon III were mixed together a Lightnin Labmaster mixer LIU10F (135 Mt. Read Blvd., Rochester, N.Y.). Tween 40 and the fragrance were mixed separately in a small container using a spatula. The fragrance/Tween 40 mixture was then mixed into the mixture containing water, glycerin, and the other ingredients identified above. Hispagel 200 and Sepigel 501 were then added in sequence to the resulting combination in an Ultra-Turrax T50 Basic high-shear homogenizer (IKA® Works, 2635 Northchase Pkwy. SE, Wilmington, N.C. 28405). Finally, pH of the formulation was adjusted by adding sodium citrate until a pH of 6.0 was achieved, as measured using a SevenMulti pH meter (Mettler-Toledo, 1900 Polaris Parkway, Columbus, Ohio, 43240).

### Example 2

### Representative Example of a Personal-Care Composition

An exemplary personal-care composition having the ingredients and proportions identified below was prepared:

| Component | Weight % | Supplier | Address |
|---|---|---|---|
| Water | 67.1 | N/A | N/A |
| Emulgade CM | 20.0 | Cognis | 300 Brookside Ave, |
| | | | Ambler, PA 19002 |
| Glycerin (99.7% | 4.0 | Glenn Corp. | 4886 Highway 61 N, |
| USP) | | | St. Paul, MN 55110 |
| Pentavitin | 4.0 | Pentapharm/ | 20 Glover Ave, |
| (saccharide | | CenterChem | Norwalk, CT 06850 |
| isomerate) | | | |
| Sepigel 501 | 2.5 | Seppic | 30, Two Bridges |
| | | | Road, Fairfield, New |
| | | | Jersey 07004 |
| Panthenol | 1.0 | Sigma-Aldrich | 3050 Spruce Street, |
| | | | St. Louis, MO 63103 |
| Paragon III | 0.5 | McIntyre Group | 24601 Governors |
| | | | Highway, University |
| | | | Park, IL 60466 |
| Keltrol CG | 0.3 | CPKelco | 1000 Parkwood |
| (Xanthan Gum) | | | Circle, Atlanta, GA |
| | | | 30339 |
| Fragrance | 0.2 | | |
| Tween 40 | 0.2 | Uniquema | 76 East 24^{th} St, |
| | | | Paterson, NJ 07544 |
| Sodium citrate | 0.2 | Sigma-Aldrich | 3050 Spruce Street, |
| 20% | | | St. Louis, MO 63103 |

The recited proportion of xanthan gum was dispersed in water by thoroughly mixing the material in a Lightnin Labmaster mixer LIU10F (135 Mt. Read Blvd., Rochester, N.Y.) at a setting of 400 rpm until the gum was fully hydrated (approximately an hour). Glycerin, Emulgade CM, and Pentavitin, followed by panthenol and Paragon III were then mixed into the xanthan-gum/water mixture. Sepigel 501 was then added to the combination and homogenized using a high shear mixer (Ultra-Turrax T50 Basic, IKA®) Works, 2635 Northchase Pkwy. SE, Wilmington, N.C. 28405). Tween 40 and the fragrance were mixed separately in a small container using a spatula. The fragrance/Tween 40 mixture was then mixed into the combination. Finally, pH of the formulation was adjusted by adding sodium citrate until a pH of 6.0 was achieved, as measured using a SevenMulti pH meter (Mettler-Toledo, 1900 Polaris Parkway, Columbus, Ohio, 43240).

### Example 3

### Representative Example of a Personal-Care Composition

An exemplary personal-care composition was prepared having the ingredients and proportions identified below was prepared:

| Component | Weight % | Supplier | Address |
|---|---|---|---|
| Water | 73.0 | N/A | N/A |
| Glycerin (99.7% | 5.0 | Glenn Corp. | 4886 Highway 61 N, |
| USP) | | | St. Paul, MN 55110 |
| Cognis IPP | 5.0 | Cognis | 300 Brookside Ave, |
| | | | Ambler, PA 19002 |
| Arlacel 165 | 3.0 | Uniquema | 76 East 24^{th} St, |
| | | | Paterson, NJ 07544 |
| Petrolatum (Super | 3.0 | Crompton | 771 Old Saw Mill |
| White Protopet) | | | River Road, |
| | | | Tarrytown, NY 10531 |
| Lipex 512 (Shea | 2.0 | Jarchem Industries | 414 Wilson Ave, |
| Butter) | | | Newark, NJ 07105 |
| BioVera Oil (Aloe | 2.0 | BioChemical | 498 Kingston Road, |
| Vera) | | International | Satellite Beach, FL 32937 |
| DC 200 Fluid, 100 cst | 2.0 | Dow Corning | PO Box 994, 2200 |
| | | | West Salzburg Road, |
| | | | Midland, MI 48686 |
| Corpure Avocado | 1.0 | Croda | 7 Century Drive, |
| (Avocado Oil) | | | Parsippany, NJ |
| | | | 07054 |
| Cetyl Alcohol (NF) | 1.0 | Glenn Corp. | 4886 Highway 61 N, |
| | | | St. Paul, MN 55110 |
| Emerest 2400 | 0.5 | Cognis | 300 Brookside Ave, |
| (Glyceryl Stearate) | | | Ambler, PA 19002 |
| dl-alpha Tocopherol | 0.5 | Ruger Chemical | 1515 W. Blancke |
| Acetate (Vitamin E | | | Street, Linden, NJ |
| acetate) | | | 07036 |
| Paragon III | 0.5 | McIntyre Group | 24601 Governors |
| | | | Highway, University |
| | | | Park, IL 60466 |
| Actiphyte of | 0.5 | Active Organics | 1097 Yates Street, |
| Chamomile AQ | | | Lewisville, TX 75057 |
| (Chamomile Extract) | | | |
| Ketrol CG (Xanthan | 0.2 | CPKelco | 1000 Parkwood |
| Gum) | | | Circle, Atlanta, GA |
| | | | 30339 |
| Fragrance | 0.2 | | |
| Tween 40 | 0.2 | Uniquema | 76 East 24^{th} St, |
| | | | Paterson, NJ 07544 |
| Sodium Citrate 20% | 0.2 | Sigma-Aldrich | 3050 Spruce Street, |
| solution | | | St. Louis, MO 63103 |
| Versene NA | 0.1 | Dow Chemical | PO Box 1206, |
| (Disodium EDTA) | | | Midland, MI 48642 |
| BHT | 0.1 | Universal Preserv-A- | 33 Truman Drive |
| | | Chem | South, Edison, NJ |
| | | | 08817 |

The recited proportion of xanthan gum was dispersed in water by thoroughly mixing the material in a Lightnin Labmaster mixer LIU10F (135 Mt. Read Blvd., Rochester, N.Y.) at a setting of 400 rpm until the gum was fully hydrated for approximately one hour. Each of the aqueous ingredients—glycerin, Paragon III, Actiphyte of Chamomile AQ, and Versene NA—were then added to the water phase. (This complete formulation is an oil-in-water (o/w) emulsion where all water-soluble ingredients are mixed separately and called “the water phase”; and the same is done for the oil-soluble ingredients denoted as the “oil phase.”) Tween 40 and the fragrance were mixed separately in a small container using a spatula. The fragrance/Tween 40 mixture was then mixed into the combination. The water phase was then heated to 76 degrees Celsius using a VWR hotplate (1310 Goshen Parkway, West Chester, Pa. 19380). Ingredients for the oil phase of the formulation were then mixed in a separate container using the Lightnin Labmaster and also heated to 76 degrees Celsius on a hotplate. The ingredients were Cognis IPP, Arlacel 165, Petrolatum, Lipex 512, BioVera Oil, DC 200 Fluid, Corpure Avocado, Cetyl Alcohol, Emerest 2400, dl-alpha tocopherol acetate, and BHT. The oil-phase mixture was then added to the water-phase mixture, both at the recited temperature of 76 degrees Celsius. The combination was mixed in a container at a setting of 400 rpm. After mixing at this speed for approximately 10 minutes, the rotational speed was increased to 470 rpm for an additional 5 minutes. Then the combination was homogenized for three minutes using a high-shear mixer (Ultra-Turrax T50 Basic, IKA® Works, 2635 Northchase Pkwy. SE, Wilmington, N.C. 28405). The combination was then allowed to cool down with the Lightnin Labmaster mixer LIU10F (135 Mt. Read Blvd., Rochester, N.Y.) set at a rotational speed of 400 rpm. Finally, pH of the formulation was adjusted by adding sodium citrate until a pH of 6.0 was achieved, as measured using a SevenMulti pH meter (Mettler-Toledo, 1900 Polaris Parkway, Columbus, Ohio, 43240).

### Example 4

### Representative Example of a Personal-Care Composition

An exemplary personal-care composition was prepared having the ingredients and proportions identified below was prepared:

| Component | % | Supplier | Address |
|---|---|---|---|
| Water | 63.7 | N/A | N/A |
| Emulgade CM | 20.0 | Cognis | 300 Brookside Ave, |
| | | | Ambler, PA 19002 |
| Glycerin (99.7% USP) | 4.0 | Glenn Corp. | 4886 Highway 61 N, |
| | | | St. Paul, MN 55110 |
| Hispagel 200 | 4.8 | Cognis | 300 Brookside Ave, |
| | | | Ambler, PA 19002 |
| Sepigel 501 | 3.2 | Seppic | 30, Two Bridges |
| | | | Road, Fairfield, New |
| | | | Jersey 07004 |
| Mackernium-007 | 1.0 | McIntyre Group | 24601 Governors |
| | | | Highway, University |
| | | | Park, IL 60466 |
| Paragon III | 0.5 | McIntyre Group | 24601 Governors |
| | | | Highway, University |
| | | | Park, IL 60466 |
| Panthenol | 0.5 | Sigma-Aldrich | 3050 Spruce Street, |
| | | | St. Louis, MO 63103 |
| Tindoerm A | 0.5 | Ciba Specialty | 4090 Premier Drive, |
| | | Chemicals | High Point, NC |
| | | | 27261 |
| Actiphyte of Aloe | 0.5 | Active Organics | 1097 Yates Street, |
| Vera extract 10 fold | | | Lewisville, TX 75057 |
| Actiphyte of Avocade | 0.25 | Active Organics | 1097 Yates Street, |
| | | | Lewisville, TX 75057 |
| Actiphyte of Jojoba | 0.25 | Active Organics | 1097 Yates Street, |
| Meal | | | Lewisville, TX 75057 |
| Fragrance | 0.2 | | |
| Tween 40 | 0.2 | Uniquema | 76 East 24^{th} St, |
| | | | Paterson, NJ 07544 |
| Tinoderm E | 0.1 | Ciba Specialty | 4090 Premier Drive, |
| | | Chemicals | High Point, NC |
| | | | 27261 |
| Sodium citrate 20% | 0.3 | Sigma-Aldrich | 3050 Spruce Street, |
| | | | St. Louis, MO 63103 |

The recited proportions of water, glycerin, Emulgade CM, Mackernium-007, Tinoderm A, Tinoderm E, Aloe Vera, Avocado, Jojoba Meal and Paragon III were mixed together a Lightnin Labmaster mixer LIU10F (135 Mt. Read Blvd., Rochester, N.Y.). Tween 40 and the fragrance were mixed separately in a small container using a spatula. The fragrance/Tween 40 mixture was then added to the previous combination. Hispagel 200 and Sepigel 501 were then added in sequence to the resulting combination in an Ultra-Turrax T50 Basic high-shear homogenizer (IKA® Works, 2635 Northchase Pkwy. SE, Wilmington, N.C. 28405). Finally, pH of the formulation was adjusted by adding sodium citrate until a pH of 6.0 was achieved, as measured using a SevenMulti pH meter (Meftler-Toledo, 1900 Polaris Parkway, Columbus, Ohio, 43240).

### Example 5

### Prophetic, Representative Appliances

One exemplary prophetic appliance is prepared in the following manner.

First, a co-extruded film is prepared composed of a central elastic layer/member sandwiched between two shielding layers/members. The central elastic layer of the cast film is made from Kraton 6638 polymer resin (Kraton 6638 is a blend of 80% by weight Kraton 1730 styrene-(ethylene-propylene)-styrene-(ethylene-propylene)tetrablock tetrablock copolymer from Kraton Polymers LLC, 7% by weight PETROTHANE NA601 polyethylene wax from Quantum Chemical Co., and 13% by weight REGALREZ 1126 tackifier from Eastman Chemical Co). The “skin” layers—that is the shielding members—formed on either side of the central elastic member are made from Achieve 3854 polypropylene available from ExxonMobil Chemical, a business having offices in Houston, Tex.

As the film is being produced (at a 75 grams per square meter basis weight prior to stretching), it is stretched by about 400% along the dimension parallel to the direction of travel of the film. To each side of the film, a necked polypropylene spunbond web, having a basis weight of 0.8 ounces per square yard, is attached. This is accomplished by first spraying a hot-melt adhesive, #2840 (available from Ato-Findley), at an add-on level of 2 grams per square meter, and heated to a temperature of 365 degrees Fahrenheit, to the side of the spunbond materials that is to contact the film. The film and nonowovens are directed to a nip between two rolls such that the spunbond material is adhesively bonded to the film, thereby creating a 3-layer substrate (the stratified film itself has 3 layers; from the perspective of the substrate, this stratified film counts as 1 layer, sandwiched between two nonwoven polypropylene webs, each counting as a single layer). The substrate is then allowed to retract, thereby gathering the spunbond material.

Once the substrate is prepared (likely having a cross-sectional appearance something like the substrate depicted in FIG. 1D), it is cut into individual pieces in the shape of hand and foot (similar to the shapes representatively depicted in FIGS. 2, 2A, 3, and 3A above). The individual pieces are then sewn together to make either a glove into which a user could insert his or her hand, or a sock into which a user could insert his or her foot,

A personal-care composition is then applied. Each of the formulations identified in Examples 1, 2, and 3 above are added to the socks and gloves. The formulations are applied to the exposed surfaces of the appliances (with the seam visible) using a syringe (4 grams) and a spatula is used to spread the formulation to cover all parts of the product. Because the elastic layer of the film is sandwiched between two shielding layers, one on either side of the elastic layer, either nonwoven/fibrous layer can be selected as the inner fibrous layer to which the formulation is applied, with the other fibrous layer then serving as the outer fibrous layer. The product is then inverted back with the seams inside and placed in air-tight bags to prevent evaporation until the time of use.

### Example 6

### Prophetic; Representative Appliances

An exemplary prophetic appliance is prepared in the following manner.

First, a laminate is prepared, with the laminate comprising a shielding member and a fibrous layer attached to the shielding member. A film composed of Basell PF-015, available from Basell Polyolefins, a business having offices in Wilmington, Del., is cast and then directed to a nip between two rolls. To one side of the film, a necked polypropylene spunbond web, having a basis weight of 0.8 ounces per square yard, is attached. This is accomplished by first spraying a hot-melt adhesive, #2840 (available from Ato-Findley), at an add-on level of 2 grams per square meter, and heated to a temperature of 365 degrees Fahrenheit, to the side of the spunbond material that is to contact the film. The film and the spunbond material are pressed together in the nip. This laminate is wound up in the form of a roll on a winder. The spunbond material is not gathered; it is substantially bonded along its length to the film that serves as a shielding member/barrier.

An elastomeric film is produced to serve as an elastic member. First, a cast elastomeric film is made from Kraton 6638 polymer resin (Kraton 6638 is a blend of 80% by weight Kraton 1730 styrene-(ethylene-propylene)-styrene-(ethylene-propylene) tetrablock copolymer from Kraton Polymers LLC, 7% by weight PETROTHANE NA601 polyethylene wax from Quantum Chemical Co., and 13% by weight REGALREZ 1126 tackifier from Eastman Chemical Co). As the film is being produced (at a 75 grams per square meter basis weight prior to stretching), it is stretched by about 400% along the dimension parallel to the direction of travel of the film.

To one side of the elastomeric film, a necked polypropylene spunbond material, having a basis weight of 0.8 ounces per square yard, is attached. This is accomplished by first spraying a hot-melt adhesive, #2840 (available from Ato-Findley), at an add-on level of 2 grams per square meter, and heated to a temperature of 365 degrees Fahrenheit, to the side of the spunbond material that is to contact the elastomeric film.

The laminate of the shielding member and nonwoven is attached to the other side of the film in a similar way. The laminate is oriented so that the shielding member—in this case a film—is brought into contact with the elastomeric film. This is accomplished by first spraying a hot-melt adhesive, #2840 (available from Ato-Findley), at an add-on level of 2 grams per square meter, and heated to a temperature of 365 degrees Fahrenheit, to the side of the shielding member of the laminate that is to contact the elastomeric film.

In summary, the elastomeric film is sandwiched between the fibrous-layer/film-shielding-member laminate on one side of the elastomeric film, and a second fibrous layer on the other side of the elastomeric film. Because neither the laminate nor the fibrous layer are stretched when attached to the elastomeric film, and because both the laminate and the fibrous layer are attached at discrete locations along the length of the elastomeric film, both the fibrous layer and the laminate are gathered when the elastomeric film is allowed to retract. A cross-section of the resulting substrate would look something like that depicted in FIG. 1C.

Once the substrate is prepared, it is cut into individual pieces in the shape of hand and foot (similar to the shapes representatively depicted in FIGS. 2, 2A, 3, and 3A above). The individual pieces are then sewn together to make either a glove into which a user could insert his or her hand, or a sock into which a user could insert his or her foot.

A personal-care composition is then applied. Each of the formulations identified in Examples 1, 2, and 3 above are added to the socks and gloves. The formulations are applied to the exposed surfaces of the appliances (with the seam visible) using a syringe (4 grams) and a spatula is used to spread the formulation to cover all parts of the product. The formulation is applied to that nonwoven/fibrous layer such that the shielding layer is interposed between the elastic layer and the formulation (i.e., the formulation is applied to that fibrous layer that is part of the laminate composed of the shielding layer and fibrous layer). The product is then inverted back with the seams inside and placed in air-tight bags to prevent evaporation until the time of use.

### Example 7

### Prophetic, Representative Appliances

An exemplary prophetic appliance is prepared in the following manner.

First, a laminate is prepared, with the laminate comprising a shielding member and a fibrous layer attached to the shielding member. A film composed of Achieve 3854 polypropylene available from ExxonMobil Chemical, a business having offices in Houston, Tex., is cast and then directed to a nip between two rolls. To one side of the film, a necked polypropylene spunbond web, having a basis weight of 0.8 ounces per square yard, is attached. This is accomplished by first spraying a hot-melt adhesive, #2840 (available from Ato-Findley), at an add-on level of 2 grams per square meter, and heated to a temperature of 365 degrees Fahrenheit, to the side of the spunbond material that is to contact the film. The film and the spunbond material are pressed together in the nip. This laminate is wound up in the form of a roll on a winder. The spunbond material is not gathered; it is substantially bonded along its length to the film that serves as a shielding member/barrier.

A composite of spaced-apart, substantially parallel elastic strands attached to meltblown fiber is produced to serve as an elastic member. First, elastic strands are extruded through orifices having an inside diameter of 0.030 inches, with a spacing of 10 orifices per inch across the width of the web being formed. The extruded strands are composed of Kraton 6638 polymer resin (Kraton 6638 is a blend of 80% by weight Kraton 1730 styrene-(ethylene-propylene)-styrene-(ethylene-propylene) tetrablock copolymer from Kraton Polymers LLC, 7% by weight PETROTHANE NA601 polyethylene wax from Quantum Chemical Co., and 13% by weight REGALREZ 1126 tackifier from Eastman Chemical Co).

After the elastic strands have been formed, meltblown fiber is attached to the elastic strands. The meltblown fiber is composed of a polypropylene grade elastomer, VistaMaxx 1100, available from ExxonMobil Chemical, a business having offices in Houston, Tex. The polymeric elastomer is heated to a temperature of 420 degrees Fahrenheit. The molten polymer is then directed though orifices having an inside diameter of 0.0145 inches, with 30 orifices per inch across the width of the web being formed. The bank of orifices is positioned such that there is a distance of 10 inches between the orifices and the web being formed. Air at a temperature of 420 degrees Fahrenheit is directed at the molten polymer strands being extruded through the orifices to attenuate the strand concurrent to the forming of a network of meltblown fiber attached to the elastic strands.

As elastic strand/meltblown composite is being produced (at a 75 grams per square meter basis weight prior to stretching), it is stretched by about 400% along the dimension parallel to the direction of the composite.

To one side of the elastomeric composite, a necked polypropylene spunbond material, having a basis weight of 0.8 ounces per square yard, is attached. This is accomplished by first spraying a hot-melt adhesive, #2840 (available from Ato-Findley), at an add-on level of 2 grams per square meter, and heated to a temperature of 365 degrees Fahrenheit, to the side of the spunbond material that is to contact the elastomeric composite.

The laminate of the shielding member and nonwoven is attached to the other side of the elastomeric composite in a similar way. The laminate is oriented so that the shielding member—in this case a film—is brought into contact with the elastomeric composite. This is accomplished by first spraying a hot-melt adhesive, #2840 (available from Ato-Findley), at an add-on level of 2 grams per square meter, and heated to a temperature of 365 degrees Fahrenheit, to the side of the shielding member of the laminate that is to contact the elastomeric composite.

In summary, the elastomeric composite is sandwiched between the fibrous-layer/film-shielding-member laminate on one side of the elastomeric composite, and a second fibrous layer on the other side of the elastomeric film. Because neither the laminate nor the fibrous layer are stretched when attached to the elastomeric composite, and because both the laminate and the fibrous layer are attached at discrete locations along the length of the elastomeric composite, both the fibrous layer and the laminate are gathered when the elastomeric composite is allowed to retract. A cross-section of the resulting substrate would look something like that depicted in FIG. 1C.

Once the substrate is prepared, it is cut into individual pieces in the shape of hand and foot (similar to the shapes representatively depicted in FIGS. 2, 2A, 3, and 3A above). The individual pieces are then sewn together to make either a glove into which a user could insert his or her hand, or a sock into which a user could insert his or her foot.

A personal-care composition is then applied. Each of the formulations identified in Examples 1, 2, and 3 above are added to the socks and gloves. The formulations are applied to the exposed surfaces of the appliances (with the seam visible) using a syringe (4 grams) and a spatula is used to spread the formulation to cover all parts of the product. The formulation is applied to that nonwoven/fibrous layer such that the shielding layer is interposed between the elastic layer and the formulation (i.e., the formulation is applied to that fibrous layer that is part of the laminate composed of the shielding layer and fibrous layer). The product is then inverted back with the seams inside and placed in air-tight bags to prevent evaporation until the time of use.

### Example 8

### Prophetic; Representative Appliances

The prophetic, exemplary appliances of Examples 5, 6, and 7 are prepared, with the exception that: the film and fibrous layers of Example 5 are thermally bonded to one another without an adhesive; the film, fibrous layer, and laminate (of a shielding layer and fibrous layer) of Example 6 are thermally bonded to one another without an adhesive; and the elastomeric composite, fibrous layer, and laminate (of a shielding layer and fibrous layer) of Example 7 are thermally bonded to one another without an adhesive. Methods disclosed elsewhere in the application, and known in the art of thermal bonding, are used to join the referenced materials to form the described appliances.

We claim:

1. A method of making an appliance adapted to transfer a composition from the appliance to the skin of a wearer of the appliance, the method comprising the steps of:
providing a formulation;
providing a film comprising an elastic layer and a shielding layer, wherein the shielding layer is impermeable to the formulation;
associating the formulation with the shielding layer so that the shielding layer is interposed between the elastic layer and the formulation.

2. The method of claim 1 wherein the formulation comprises a humectant, an ingredient adapted to occlude liquid, an emollient, an antioxidant, chelating agents, emulsifiers, surfactants, viscosity modifiers, natural moisturizing factors, antimicrobial actives, pH modifiers, enzyme inhibitors/inactivators, suspending agents, pigments, dyes, colorants, buffers, perfumes, antibacterial actives, antifungal actives, pharmaceutical actives, film formers, deodorants, opacifiers, astringents, solvents, organic acids, preservatives, drugs, vitamins, aloe vera, skin conditioners, botanical agents or some combination thereof.

3. The method of claim 1 wherein the film further comprises a second shielding layer, and wherein the elastic layer is sandwiched between the shielding layer and the second shielding layer.

4. A method of selling an appliance adapted to transfer a composition from a surface of the appliance to the skin of a wearer of the appliance, the method comprising the steps of:
participating in a business arrangement with a contract manufacturer, wherein the contract manufacturer, under said business arrangement, makes the appliance in accordance with the steps enumerated in claim 1;
receiving the appliance made by the contract manufacturer; and
selling the appliance.

5. A method of making an appliance adapted to transfer a composition from a surface of the appliance to the skin of a wearer of the appliance, the method comprising the step of:
participating in a business arrangement with a contract manufacturer, wherein the contract manufacturer, under said business arrangement, makes the appliance in accordance with the steps enumerated in claim 1.

6. A method of making an appliance adapted to transfer a composition from the appliance to the skin of a wearer of the appliance, the method comprising the steps of:
providing a formulation;
providing a film comprising an elastic layer and a shielding layer, wherein the shielding layer is impermeable to the formulation;
providing a fibrous web;
stretching the film;
directing the fibrous web and the stretched film to a nip between two rolls;
attaching the fibrous web to the shielding layer of the stretched film;
allowing the combination of the stretched film and fibrous web to retract; and
associating the formulation with the fibrous layer so that the shielding layer is interposed between the elastic layer and the formulation.

7. The method of claim 6 wherein the fibrous web is substantially unstretched when directed to the nip.

8. Them method of claim 6 wherein the fibrous web is attached to the shielding layer at discrete locations along the length of the stretched film.

9. A method of making an appliance adapted to transfer a composition from a surface of the appliance to the skin of a wearer of the appliance, the method comprising the step of:
participating in a business arrangement with a contract manufacturer, wherein the contract manufacturer, under said business arrangement, makes the appliance in accordance with the steps enumerated in claim 6.

10. The method of claim 6 wherein the fibrous layer is attached to the shielding layer thermally, ultrasonically, with adhesive, or some combination thereof.

11. A method of making an appliance adapted to transfer a composition from a surface of the appliance to the skin of a wearer of the appliance, the method comprising the steps of:
providing a formulation;
providing a film comprising an elastic layer and a shielding layer, wherein the shielding layer is impermeable to the formulation;
providing a first fibrous web;
providing a second fibrous web;
stretching the film;
directing the first fibrous web, the second fibrous web, and the stretched film to a nip between two rolls, wherein the first fibrous web faces one side of the stretched film, and wherein the second fibrous web faces the opposing side of the stretched film;
attaching the first fibrous web to one side of the stretched film and the second fibrous web to the opposing side of the stretched film;
allowing the combination of the stretched film and fibrous webs to retract.; and
associating the formulation with the fibrous layer attached to the shielding layer of the film so that the shielding layer is interposed between the elastic layer and the formulation.

12. The method of claim 11 wherein the film further comprises a second shielding layer such that the elastic layer is sandwiched between the shielding layer and the second shielding layer.

13. The method of claim 11 wherein the fibrous webs are substantially unstretched when directed to the nip.

14. Them method of claim 11 wherein the fibrous webs are attached to the film at discrete locations along the length of the stretched film.

15. A method of making an appliance adapted to transfer a composition from a surface of the appliance to the skin of a wearer of the appliance, the method comprising the step of:
participating in a business arrangement with a contract manufacturer, wherein the contract manufacturer, under said business arrangement, makes the appliance in accordance with the steps enumerated in claim 11.

16. The method of claim 11 wherein the fibrous layer is attached to the shielding layer thermally, ultrasonically, with adhesive, or some combination thereof.

17. A method of making an appliance adapted to transfer a composition from a surface of the appliance to the skin of a wearer of the appliance, the method comprising the steps of:
providing a formulation;
providing an elastic layer comprising a plurality of elastic strands;
providing a laminate comprising a first fibrous web attached to a shielding layer;
providing a second fibrous web;
stretching the elastic layer;
directing the laminate, the second fibrous web, and the stretched elastic layer to a nip between two rolls, wherein the shielding layer of the laminate faces one side of the stretched elastic layer, and wherein the second fibrous web faces the opposing side of the stretched elastic layer;
attaching the shielding layer of the laminate to one side of the stretched elastic layer and the second fibrous web to the opposing side of the stretched elastic layer;
allowing the combination of the stretched elastic layer, the laminate, and the second fibrous web; and
associating the formulation with first fibrous layer so that the shielding layer is interposed between the elastic layer and the formulation.

18. The method of claim 17 wherein the laminate and the second fibrous layer are substantially unstretched when directed to the nip.

19. The method of claim 17 wherein the fibrous webs are attached to the elastic layer at discrete locations along the length of the stretched elastic layer.

20. The method of claim 17 wherein the elastic layer is liquid permeable.

21. A method of making an appliance adapted to transfer a composition from a surface of the appliance to the skin of a wearer of the appliance, the method comprising the step of:
participating in a business arrangement with a contract manufacturer, wherein the contract manufacturer, under said business arrangement, makes the appliance in accordance with the steps enumerated in claim 17.

22. The method of claim 17 wherein the fibrous layer is attached to the shielding layer thermally, ultrasonically, with adhesive, or some combination thereof.

23. A method of making an appliance adapted to transfer a composition from a surface of the appliance to the skin of a wearer of the appliance, the method comprising the steps of:
providing a formulation;
providing an elastic layer comprising an elastic film;
providing a laminate comprising a first fibrous web attached to a shielding layer;
providing a second fibrous web;
stretching the elastic layer;
directing the laminate, the second fibrous web, and the stretched elastic layer to a nip between two rolls, wherein the shielding layer of the laminate faces one side of the stretched elastic layer, and wherein the second fibrous web faces the opposing side of the stretched elastic layer;
attaching the shielding layer of the laminate to one side of the stretched elastic layer and the second fibrous web to the opposing side of the stretched elastic layer;
allowing the combination of the stretched elastic layer, the laminate, and the second fibrous web to retract; and
associating the formulation with first fibrous web so that the shielding layer is interposed between the elastic layer and the formulation.

24. The method of claim 23 wherein the laminate and the second fibrous web substantially unstretched when directed to the nip.

25. The method of claim 23 wherein the fibrous webs are attached to the elastic layer at discrete locations along the length of the stretched elastic layer.

26. The method of claim 1, 6, 11, 17, and 23 wherein the appliance is a sock, glove, sleeve, or patch.
